(19) United States
(12) Patent Application Publication
(10) Pub. No.: US 2017/0151283 A1
(43) Pub. Date: Jun. 1, 2017

(54) COMPOSITIONS AND METHODS FOR TREATING ANTIBODY RESISTANCE

(71) Applicant: THE TRUSTEES OF THE UNIVERSITY OF PENNSYLVANIA, Philadelphia, PA (US)

(72) Inventor: Daniel J. POWELL, Bala Cynwyd, PA (US)

(21) Appl. No.: 15/311,399

(22) PCT Filed: May 22, 2015
(86) PCT No.: PCT/US15/32291

§ 371 Date: Nov. 15, 2016

Related U.S. Application Data
(60) Provisional application No. 62/002,775, filed on May 23, 2014.

(51) Int. Cl. A61K 35/17; C07K 14/725; C07K 16/32; A61K 39/395; C07K 14/705; C07K 16/28; C07K 14/735; C12N 5/0783

(52) U.S. Cl. CPC A61K 35/17; C07K 14/70535; C07K 14/7051; C12N 5/0636; A61K 39/3955; C07K 14/70521; C07K 16/2893; C07K 16/2863; C07K 16/2887; C07K 16/32; C07K 2319/03; C12N 2510/00; C07K 2319/70; A61K 38/00

(57) ABSTRACT

The invention provides compositions and methods for overcoming poor response to antibody therapy, for example, antibody resistance. The invention also relates to at least one immune receptor (IR) specific to the Fc receptor, vectors comprising the same, and recombinant T cells comprising the Fc immune receptor. The invention also includes methods of administering a modified T cell expressing an immune receptor that comprises a Fc binding domain.

### CROSS-REFERENCE TO RELATED APPLICATIONS

This application claims priority to U.S. Provisional Application No. 62/002,775, filed on May 23, 2014, which is hereby incorporated by reference in its entirety herein.

### STATEMENT REGARDING FEDERALLY SPONSORED RESEARCH OR DEVELOPMENT

This invention was made with government support under CA168900 awarded by the National Institute of Health. The government has certain rights in the invention.

### BACKGROUND OF THE INVENTION

Adoptive transfer of T cells is a powerful approach for treating patients with advanced malignancies. However, there are some limitations to such treatments, such as persistence of the cells, immunogenicity, and toxicities associated with on- and off-target activation.

Targeted therapies utilizing monoclonal antibodies (mAbs) are now the major class of successful therapeutics for treating a variety of malignancies. Antibody therapy is based on the development of monoclonal antibodies (mAb) directed against tumor cell surface antigens (TAA). Mechanistically, mAbs can have direct antitumoral activity but often their effectiveness relies upon antibody-dependent cellular cytotoxicity (ADCC). In ADCC immune effector cells, mainly natural killer cells (NK cells), bind via their Fc receptor (FcγRIII, CD16), to the Fc portion of a therapeutic mAb. This leads to the activation of NK cells, the release of their cytotoxic granules, and subsequent lysis of the antibody-bound cancer cell. Despite the great promise of antibody-based therapies, several limitations have emerged including, but not limited to, a high cost and insufficient drug action demonstrated by frequent relapses observed in mAbs-treated cancer patients. This is all evidence that the bioactivity of these types of therapeutics is still suboptimal.

Given that NK cells are the main effector cells in ADCC, several investigators have explored the use of adoptively transferred allogeneic NK cells for treating patients with cancer and other strategies focused on improving NK cells function. It has been shown that NK cell-mediated ADCC response against tumor targets can be promoted by administration of mAbs to tumor-associated antigens combined with cytokine-based immunotherapies. Indeed, several cytokines enhance NK cell response to mAb treatment, and show very promising results in pre-clinical studies. However, despite the large number of studies that demonstrate the ability of NK cells to kill tumor target cells in vitro and in vivo in animal models, the clinical efficacy of activated NK cells in cancer therapy has proven effective only in a few cases. Moreover, toxicity of systemic cytokine administration and cytokine-activated NK cell apoptosis are important limitations of cytokine-mediated (and NK adoptive) immunotherapies for cancer treatment. It has also been shown that tumor cells express inhibitory molecules, and that the engagement of inhibitory receptors by NK cells inhibits their anti-tumor antibody mediated response. Indeed inhibitory NK receptors have been hypothesized to represent an important mechanism of specific immune suppression which may inhibit NK activation by antigen bound antibody, therefore limiting ADCC.

Therefore, a need exists for the development of better methods of increasing response to antibody therapies or alternative therapies. The present invention satisfies this need.

### SUMMARY OF THE INVENTION

As described herein, the present invention includes compositions and methods for overcoming a sub-optimal response to antibody therapy. One aspect of the invention includes an isolated nucleic acid sequence encoding an immune receptor (IR), wherein the isolated nucleic acid sequence comprises a human nucleic acid sequence of a Fc receptor (Fc) binding domain and a nucleic acid sequence of an intracellular domain of a costimulatory molecule.

Another aspect of the invention includes a vector comprising an isolated nucleic acid sequence encoding an immune receptor (IR), wherein the isolated nucleic acid sequence comprises a human nucleic acid sequence of a Fc receptor (Fc) binding domain and a nucleic acid sequence of an intracellular domain of a costimulatory molecule.

In yet another aspect, the invention includes an isolated immune receptor (IR) comprising a Fc receptor (Fc) binding domain and an intracellular domain of a costimulatory molecule.

In another aspect, the invention includes a modified T cell comprising an isolated immune receptor (IR) comprising a Fc receptor (Fc) binding domain and an intracellular domain of a costimulatory molecule.

In yet another aspect, the invention includes a pharmaceutical composition comprising the modified T cell described herein and a pharmaceutically acceptable carrier.

In still another aspect, the invention includes use of the modified T cell described herein in the manufacture of a medicament for the treatment of an immune response in a subject in need thereof.

In another aspect, the invention includes a method of treating a disease or condition associated with resistance to an antibody-mediated therapy in a subject comprising administering to the subject a therapeutically effective amount of a pharmaceutical composition comprising the modified T cell described herein. In yet another aspect, the invention includes a method of treating a condition in a subject, comprising administering to the subject a therapeutically effective amount of a pharmaceutical composition comprising the modified T cell described herein. In still another aspect, the invention includes a method for stimulating a T cell-mediated immune response to a target cell or tissue in a subject comprising administering to a subject a therapeutically effective amount of a pharmaceutical composition comprising the modified T cell described herein. In various embodiments of the above aspects or any other aspect of the invention delineated herein, the Fc binding domain is selected from the group consisting of CD64, CD32, CD16, a fragment thereof, and any combination thereof. In another embodiment, the Fc binding domain is capable of binding an antibody.

In one embodiment, the intracellular domain comprises at least one signaling domain of the co-stimulatory molecule. In another embodiment, the intracellular domain comprises at least one signaling domain selected from the group consisting of CD3, CD28, a fragment thereof, and any combination thereof.

In another embodiment, the co-stimulatory molecule is selected from the group consisting of CD3, CD27, CD28, ICOS, 4-1BB, PD-1, T cell receptor (TCR), co-stimulatory molecules, any derivative or variant of these sequences, any synthetic sequence that has the same functional capability, and any combination thereof.

In another embodiment, the isolated nucleic acid sequence or immune receptor further comprises a nucleic acid sequence of a transmembrane domain.

In one embodiment, the modified T cell further comprises an antibody bound to the Fc binding domain, wherein the antibody binds a target cell.

In another aspect, the invention includes a method for overcoming resistance to an antibody-mediated therapy in a subject, the method comprising administering to the subject an effective amount of a modified T cell comprising an immune receptor (IR), wherein the immune receptor comprises a Fc receptor (Fc) binding domain and an intracellular domain of a costimulatory molecule, thereby overcoming resistance to the antibody-mediated therapy in the subject.

In one aspect, the invention includes a method of treating a tumor in a mammal, the method comprising administering to the subject an effective amount of a genetically modified cell comprising an immune receptor (IR), wherein the immune receptor comprises a Fc receptor (Fc) binding domain and an intracellular domain of a costimulatory molecule.

In one embodiment, the administration comprises administering an antibody for a target cell prior to administering the effective amount of the modified T cell. In yet another embodiment, the administration comprises binding the modified T cell to an antibody with specificity for a target cell.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments, which are presently preferred. It should be understood, however, that the invention is not limited to the precise arrangements and instrumentalities of the embodiments shown in the drawings.

FIG. 1 is an illustration of a novel platform for antibody directed cellular cytotoxicity.

FIG. 2 is an illustration of an effector cell with an immune receptor. Human CD64 Fc-binding immune receptor, (h)CD64FcIR, includes an extracellular portion of human CD64 fused to CD28 transmembrane and T cell signaling domains.

FIG. 3 is an illustration of two Fc immune receptor constructs. A schematic representation is shown of avidin based immune receptor gene constructs containing extracellular avidin as a monomer (mcAV) or dimer (dcAv) fused to the human CD3z cytosolic domain alone (BBIR-z) or in combination with the CD28 co-stimulatory module (BBIR-28z).

FIG. 4 is a panel of flow diagrams showing expression of Fc immune receptors with CD3zeta intracellular signaling domain or CD28-CD3zeta intracellular signaling domains. BBIR expression (open histograms) is detected via GFP expression for mcAv constructs, or anti-avidin antibody for dcAV constructs. Staining of the cells is shown 5 days after transduction with lentivirus and a comparison to untransduced T cells (grey filled histograms). Percent CAR transduction is indicated.

FIG. 5 is a panel of flow diagrams showing Fc immune receptors expressed on the surface of T cells bind antibodies. Biotin re-directed dcAV, but not mcAV.BBIR T cells secrete IFNγ in response to plate-bound biotinylated, but not non-biotinylated, antibody or scFv (10 ng) in overnight culture. Concentration of IFNγ is expressed as mean±SEM in pg/ml from triplicate wells.

FIG. 6 is a panel of flow diagrams showing binding of antigen specific antibodies to the Fc immune receptors on the T cells. The upper graphs show that dcAv.BBIR-z and dcAv.BBIR-28z transduced T cells specifically react against immobilized biotinylated-IgG1. Biotin re-directed dcAv.BBIR-z and dcAv.BBIR-28z T cells secrete IFNγ in response to plate-bound biotinylated antibody in an overnight culture at the lowest concentration of ing/well. dcAv.BBIR-z, dcAv.BBIR-28z T cells or control GFP cells (10⁵ cells/well) are incubated with plate-immobilized antibody at a concentration range 0-100 ng/well. Concentration of IFNγ is expressed in pg/ml (means±SEM; n=3).

FIG. 7 is an illustration of T cell-mediated cytotoxicity using antibody armed Fc immune receptor T cells.

FIG. 8 is an illustration of “painting” or coating target cells with antibody and administration of Fc immune receptor T cells.

FIG. 9 is a panel of flow diagrams showing expression and binding of antigen specific antibodies to the Fc immune receptors on the T cells. The target cells display antigens that specifically bind the antibody bound Fc immune receptor. BBIRs respond against immobilized human mesothelin protein when redirected with biotinylated anti-mesothelin molecules (Bio-P4scFv and Bio-K1 Ab). The dcAv.BBIR-z, dcAv.BBIR-28z T cells or control GFP cells (10⁵ cells/well) are incubated with 10 ng of plate-immobilized mesothelin and with either biotinylated or not, anti-mesothelin antibodies or scFvs (0.1 μg/ml). Overnight culture supernatants are analyzed for human IFNγ cytokine by ELISA. Data represent the means±SD for 3 different experiments.

FIG. 10 is a panel of flow diagrams showing binding of Fc immune receptors on the T cells to target cells bound with antibodies. The target cells display antigens that specifically bind the antibodies. The antibodies, in turn, bind the Fc immune receptors expressed on the T cells. Retention of specific biotinylated molecules on the BBIR T cell surface assessed by flow cytometry is shown. BBIR⁺ T cells incubated with 10 ng biotinylated reagents, Biotin-APC or biotinylated-scFvP4 (open histograms), are compared to untransduced control T cells (grey).

FIG. 11 is a graph showing IFN-γ secreted by T cells after activation of antibody armed Fc immune receptors.

FIG. 12 is a graph showing IFN-γ secreted by T cells after activation of Fc immune receptors by binding antibody coated target cells.

FIG. 13 is a graph showing levels of IFN-γ, TNF, IL-2, MIP1, IL-4 and IL-10 of T cells with activated antibody armed Fc immune receptors and Fc immune receptors bound to antibody coated target cells. BBIRs exhibit effector functions in the presence of free biotin at physiological concentrations. BBIR T cells incubated overnight with Bio-K1 Ab or Bio-P4 painted immobilized mesothelin protein or with plate-bound biotinylated Abs in the presence of the indicated concentration of biotin are shown. The concentration of IFNγ is expressed as mean±SEM in pg/ml from triplicate wells.

FIG. 14 is a panel of flow diagrams showing expression of Fc immune receptor and IFN-γ (left diagram) or TNF-α (right diagram) positive T cells. BBIR T cells respond against painted EpCAM on A1847 cancer cell surface. dcAv.BBIR-28z⁺ or control GFP⁺ T cells (10⁵) are cultured with an equal number of human A1847 unlabeled or labeled with biotinylated anti-EpCAM Ab (0 up to 1000 ng). After overnight incubation, cell-free supernatants are analyzed for human IFNγ by ELISA. Surface EpCAM expression (open histograms) is detectable after labeling with different concentrations of biotinylated EpCAM Ab was evaluated by flow cytometry. The correlation of detectable Bio-EpCAM mean fluorescence intensity (MFI) on EpCAM⁺ tumors is plotted vs. the production of IFNγ by BBIR-28z T cells when co-cultured with labeled cancer cells.

FIG. 15 is a panel of graphs showing specific lytic activity of anti-EpCAM antibody armed Fc immune receptor T cells against EpCAM expressing tumors. Data are shown as mean±SEM. BBIR⁺ T cells exhibit effector functions against painted cell surface tumor antigens in the presence of antigen-specific biotinylated antibodies. Results depict the mean±SEM of triplicate wells.

FIG. 16 is a panel of graphs showing antibody mediated immune recognition of EpCAM and Her2 expressing cancer cells by CD64 Fc immune receptors. Primary human T cells are transduced to express P4-28z CAR or dcAv. BBIR-28z T cells are co-cultured with Cr⁵¹-labeled A1847 cells with painted mesothelin (Bio-K1) or EpCAM (Bio-EpCAM) for 17 hrs at the indicated effector to target ratio. Percent specific target cell lysis is calculated as (experimental−spontaneous release)÷(maximal−spontaneous release)×100. Data represent the means±SD for 3 different experiments. *P≦0.005 comparing BBIR⁺/Bio-K1 and BBIR⁺/Bio-IgG1 T cells. **P≦0.005 comparing BBIR⁺ and P4 CAR⁺ T cells and ***P≦0.005 comparing BBIR⁺/Bio-EpCAM and BBIR⁺/Bio-IgG1 T cells. The difference between the cytotoxic activity is statistically significant at the given E:T ratio.

FIG. 17 is a panel of graphs showing antibody mediated immune recognition of different EpCAM and Her2 expressing cancer cells by CD64 Fc immune receptors. The dcAv.BBIR-28z⁺ T lymphocytes produce inflammatory cytokines in response to painted A1847 tumor cells with biotinylated antibodies, anti-mesothelin (Bio-K1) and/or anti-EpCAM (Bio-EpCAM). The BBIR⁺ T cells produce equal levels of IFNγ, and Th1 cytokines in response to the painted A1847 cells in comparison with conventional anti-mesothelin P4-28z CAR⁺ T cells. Overnight culture supernatants are analyzed for human IFNγ cytokine by ELISA. The concentration of IFNγ is expressed as mean±SEM in pg/ml from triplicate wells.

FIG. 18 is a panel of graphs showing the levels of IFN-γ of T cells with antibody armed Fc immune receptors for EpCAM and Her2 after immune recognition of different EpCAM and Her2 expressing cancer cells.

FIG. 19 is a panel of graphs showing lytic activity of Fc immune receptor T cells that are armed with antibody or exposed to antibody coated target cells.

FIG. 20 is a panel of graphs showing that Her2 antibody-mediated tumor lysis is significantly enhanced in the presence of Fc immune receptors.

FIG. 21 is a panel of images with one image showing a schematic of the Her2 in vivo assay and another image showing mouse imaging of injected T cells.

FIG. 22 a panel of images showing tumor diameter and CD3+ cells in mice bearing Her2 positive tumors and injected with Fc immune receptor T cells.

FIG. 23 is a panel of graphs showing expression of Fc immune receptors with different extracellular domains in primary T cells.

FIG. 24 is an illustration showing functional effector activity of antibody armed Fc immune receptor T cells with different extracellular domains in the immune receptor.

FIG. 25 is a panel of graphs showing binding of Fc immune receptors to different extracellular domains to antibodies.

FIG. 26 is a graph showing IFN-γ levels secreted by T cells displaying antibody armed Fc immune receptors with different extracellular domains, CD64, CD32 and CD16.

FIG. 27 is a panel of images showing functional effector activity of Fc immune receptor T cells with different extracellular domains in the immune receptor. The Fc immune receptor T cells specifically bind antibody coated target cells and secrete IFN-γ.

FIG. 28 is a panel of graph showing specific binding of Fc immune receptors with different extracellular domains to antibodies and Her2 expressing tumor cells.

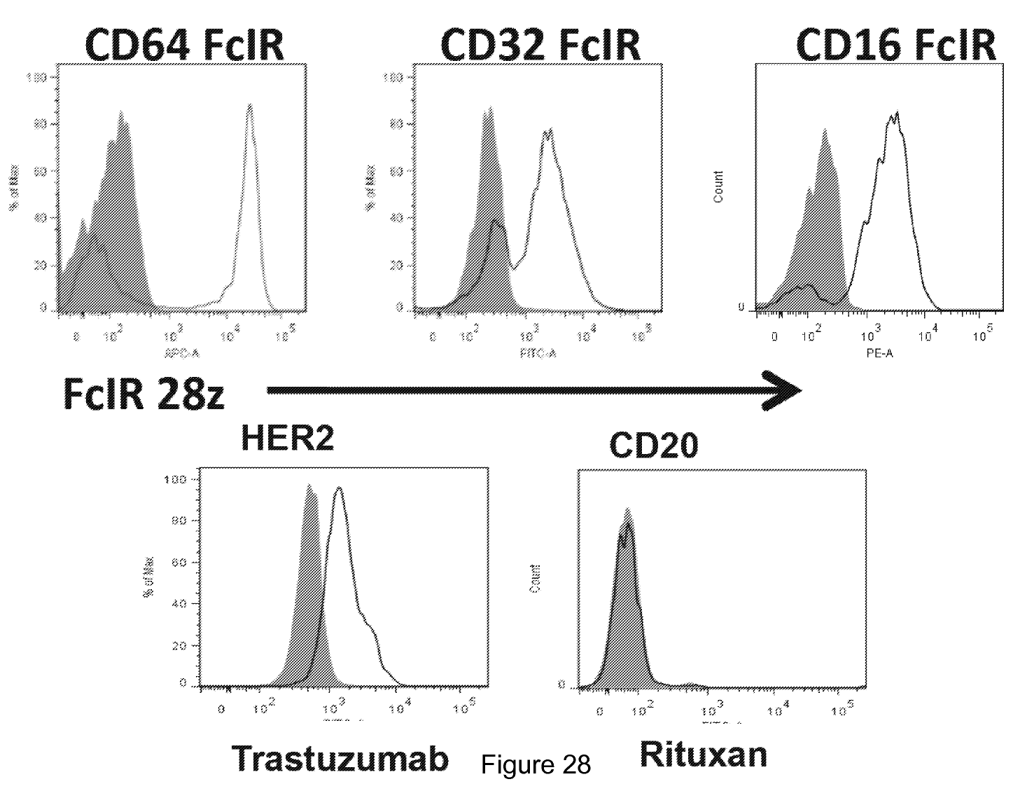

FIG. 29 is a panel of graphs showing that tumor-associated antigen specific antibody armed Fc immune receptor T cells mediate tumor lysis. Shown are percentages of lysis of target cells by chromium release at varying effector effector/target cell ratios. Data are shown as mean±SEM.

FIG. 30 is a panel of graphs showing that control antibody armed Fc immune receptor T cells do not mediate tumor lysis. Shown are percentages of lysis of target cells by chromium release at varying effector effector/target cell ratios. Data are shown as mean±SEM.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, in some instances ±5%, in some instances ±1%, and in some instance ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions.

The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The an antibody in the present invention may exist in a variety of forms where the antigen binding portion of the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to at least one portion of an intact antibody and refers to the antigenic determining variable regions of an intact antibody. Examples of antibody fragments include, but are not limited to, Fab, Fab′, F(ab′)2, and Fv fragments, linear antibodies, sdAb (either V_{L} or V_{H}), camelid V_{HH} domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “high affinity” as used herein refers to high specificity in binding or interacting or attraction of one molecule to a target molecule.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “limited toxicity” as used herein, refers to the peptides, polynucleotides, cells and/or antibodies of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, glioma, and the like.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, for example, one or more amino acid residues within the CDR regions of an antibody of the invention can be replaced with other amino acid residues from the same side chain family and the altered antibody can be tested for the ability to bind FRβ using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to, TCR, CD3 zeta, CD3 gamma, CD3 delta, CD3 epsilon, CD86, common FcR gamma, FcR beta (Fc Epsilon R1b), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, MHC class I molecule, BTLA and a Toll ligand receptor other co-stimulatory molecules described herein, any derivative, variant, or fragment thereof, any synthetic sequence of a co-stimulatory molecule that has the same functional capability, and any combination thereof.

A “co-stimulatory signal”, as used herein, refers to a signal, which in combination with a primary signal such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “Fc binding domain” or “Fc receptor binding domain” refers to a domain of the Fc immune receptor that is capable of binding an antibody.

The term “Fc immune receptor,” “Fc binding immune receptor,” “FcIR” as used herein, refers to an engineered receptor including an extracellular domain comprising a Fc-receptor or fragment thereof, and an intracellular domain of a costimulatory molecule. In one embodiment, the extracellular domain of the Fc immune receptor is capable of binding an antibody.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

The guide nucleic acid sequence may be complementary to one strand (nucleotide sequence) of a double stranded DNA target site. The percentage of complementation between the guide nucleic acid sequence and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The guide nucleic acid sequence can be at least 10, 11, 12, 13, 14, 15, 16, 17, 18, 19, 20, 21, 22, 23, 24, 25, 26, 27, 28, 29, 30, 31, 32, 33, 34, 35 or more nucleotides in length. In some embodiments, the guide nucleic acid sequence comprises a contiguous stretch of 10 to 40 nucleotides. The variable targeting domain can be composed of a DNA sequence, a RNA sequence, a modified DNA sequence, a modified RNA sequence (see for example modifications described herein), or any combination thereof.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

The term “intracellular domain” refers to the internal portion or cytoplasmic domain of the Fc immune receptor.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a virus within the genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (13) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

As used herein, “tumor cells” or simply “tumor” refers to the tumor tissue as a whole, including different cell types that are present in a tumor environment.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention includes compositions for and methods of use of immune receptors. For example, the invention includes a Fc receptor (Fc) immune receptor that includes a Fc binding domain that is capable of binding an antibody.

### Fc Immune Receptor

In one aspect, the invention includes an isolated immune receptor (IR) comprising a Fc receptor (Fc) binding domain, and an intracellular domain of a costimulatory molecule.

In another aspect, the invention includes an isolated nucleic acid sequence encoding an immune receptor (IR), wherein the isolated nucleic acid sequence comprises a human nucleic acid sequence of a Fc receptor (Fc) binding domain and a nucleic acid sequence of an intracellular domain of a costimulatory molecule.

In another aspect, the invention includes a modified T cell comprising an isolated nucleic acid sequence encoding an immune receptor (IR), wherein the isolated nucleic acid sequence comprises a human nucleic acid sequence of a Fc receptor (Fc) binding domain and a nucleic acid sequence of an intracellular domain of a costimulatory molecule.

In yet another aspect, the invention includes a modified T cell comprising an isolated immune receptor (IR) comprising a Fc receptor (Fc) binding domain and an intracellular domain of a costimulatory molecule. The invention also includes a population of cells, such as T cells, comprising the isolated nucleic acid sequence encoding the IR as described herein. A population of cells, such as T cells, comprising the IR as described herein is also included in the invention.

Extracellular Domain

The Fc immune receptor comprises an extracellular domain with a Fc binding domain. One of the most important mechanisms by which IgG antibodies engage the cellular immune system is via interaction of the Fc domain with Fcγ receptors (FcγRs). The human FcγR family contains six known members in three subgroups, including FcγRI (CD64), FcγRIIa,b,c (CD32a,b,c) and FcγRIIIa,b (CD16a,b), expressed by various effector cells of the immune system, including macrophages, neutrophils, dendritic cells and natural killer (NK) cells. The latter cell type is the main agent of antibody-dependent, cell-mediated cytotoxicity (ADCC). These cells can be recruited and activated through the interaction between FcγRIIIa and the Fc region, leading to the formation of an immunological synapse, the release of perforin/granzyme and the establishment of the Fas/FasL interaction, both leading to apoptosis of the target cells. The other cell types phagocytose target cells.

In some embodiments, the Fc binding domain comprises one or more domains from a Fc receptor. Nonlimiting examples include CD16, CD32 and CD64. In one embodiment, the Fc binding domain is selected from the group consisting of CD64, CD32, CD16, a fragment thereof, and any combination thereof. In another embodiment the Fc binding domain is capable of binding an antibody.

Intracellular Domain

The intracellular domain or otherwise the cytoplasmic domain of the IR of the invention is responsible for activation of at least one of the normal effector functions of the immune cell in which the IR has been placed in. The term “effector function” refers to a specialized function of a cell. Effector function of a T cell, for example, may be cytolytic activity or helper activity including the secretion of cytokines. Thus the term “intracellular domain” refers to the portion of a protein which transduces the effector function signal and directs the cell to perform a specialized function. While usually the entire intracellular domain can be employed, in many cases it is not necessary to use the entire chain. To the extent that a truncated portion of the intracellular domain is used, such truncated portion may be used in place of the intact chain as long as it transduces the effector function signal. The term intracellular domain is thus meant to include any truncated portion of the intracellular domain sufficient to transduce the effector function signal.

Examples of intracellular domains for use in the IR of the invention include the cytoplasmic sequences of the T cell receptor (TCR) and co-receptors, such as CD3, CD27, CD28, ICOS, 4-1BB, PD-1, that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the IR of the invention comprises a cytoplasmic signaling sequence derived from CD3-zeta.

In a preferred embodiment, the intracellular domain of the IR is designed to comprise the CD3-zeta signaling domain by itself or combined with any other desired cytoplasmic domain(s) useful in the context of the IR of the invention. For example, the cytoplasmic domain of the IR can comprise a CD3 zeta chain portion and a costimulatory signaling region. In one embodiment, the intracellular domain comprises at least one signaling domain selected from the group consisting of CD3, CD28, a fragment thereof, and any combination thereof.

The signaling domain refers to a portion of the IR comprising the intracellular domain of a costimulatory molecule. A costimulatory molecule is a cell surface molecule other than an antigen receptor or its ligands that is required for an efficient response of lymphocytes to an antigen. Examples of the intracellular domain include one or more molecules or receptors including, but are not limited to, TCR, CD3 zeta, CD3 gamma, CD3 delta, CD3 epsilon, CD86, common FcR gamma, FcR beta (Fc Epsilon R1b), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combination thereof. In one embodiment, the co-stimulatory molecule is selected from the group consisting of CD3, CD27, CD28, ICOS, 4-1BB, PD-1, T cell receptor (TCR), co-stimulatory molecules, any fragment, derivative or variant of these sequences, any synthetic sequence that has the same functional capability, and any combination thereof.

The intracellular domain of the Fc immune receptor may include one or more signaling domains of a co-stimulatory molecule linked to each other in a random or specified order. Optionally, a short oligo- or polypeptide linker, for example, between 2 and 10 amino acids in length may form the linkage. A glycine-serine doublet provides a particularly suitable linker.

Transmembrane Domain

In one embodiment, the Fc immune receptor further comprises a transmembrane domain. In embodiments comprising an isolated nucleic acid sequence, the isolated nucleic acid sequence further comprises a nucleic acid sequence encoding a transmembrane domain. With respect to the transmembrane domain, in various embodiments, the IR is designed to comprise a transmembrane domain that is fused to the extracellular domain of the IR. In one embodiment, the transmembrane domain that naturally is associated with one of the domains in the IR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CDS, CD8, CD9, CD16, CD22, CD33, CD37, CD64, CD80, CD86, CD134, CD137, CD154. In some instances, a variety of human hinges can be employed as well including the human Ig (immunoglobulin) hinge.

In one embodiment, the transmembrane domain may be synthetic, in which case it will comprise predominantly hydrophobic residues such as leucine and valine. In one aspect a triplet of phenylalanine, tryptophan and valine will be found at each end of a synthetic transmembrane domain. Optionally, a short oligo- or polypeptide linker, between 2 and 10 amino acids in length may form the linkage between the transmembrane domain and the cytoplasmic signaling domain of the IR. A glycine-serine doublet provides a particularly suitable linker.

Spacer Domain

The Fc immune receptor may include a spacer domain.

As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

Antibody

In some embodiments, the Fc binding domain of the immune receptor is bound to an antibody. Thus, the immune receptor comprises an Fc receptor (Fc) binding domain, an intracellular domain of a costimulatory molecule and an antibody. The antibody should be construed to include a synthetic antibody, a human antibody, a humanized antibody, a single domain antibody, a single chain variable fragment, and any combination or fragment thereof. In some instances, it is beneficial that the antibody is derived from the same species in which the immune receptor will ultimately be used in. For example, for use in humans, it may be beneficial that the antibody is a human antibody or fragment thereof.

For in vivo use of antibodies in humans, it may be preferable to use human antibodies. Human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety. A human antibody can also be an antibody wherein the heavy and light chains are encoded by a nucleotide sequence derived from one or more sources of human DNA.

Alternatively, in some embodiments, a non-human antibody may be humanized, where specific sequences or regions of the antibody are modified to increase similarity to an antibody naturally produced in a human. In one embodiment, the antigen binding domain portion of the antibody is humanized.

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain is substituted with the corresponding sequence from a nonhuman species. In practice, humanized antibodies are typically human antibodies in which some CDR residues and possibly some FR residues are substituted by residues from analogous sites in rodent antibodies. Humanization of antibodies can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized where retention of high affinity binding for the target antigen and other favorable biological properties are retained. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A “humanized” antibody retains a similar antigenic specificity as the original antibody. However, using certain methods of humanization, the affinity and/or specificity of binding of the antibody for human CD3 antigen may be increased using methods of “directed evolution,” as described by Wu et al., J. Mol. Biol., 294:151 (1999), the contents of which are incorporated herein by reference herein in their entirety.

In one embodiment, the antibody is a synthetic antibody, human antibody, humanized antibody, single domain antibody, single chain variable fragment, or an antigen-binding fragment thereof.

The antibody may be bound to the Fc immune receptor in vitro, prior to administration into a subject, or it may be pre-administered to the subject to bind its target in vivo and then bind the Fc immune receptor in vivo.

In one embodiment, the antibody bound to the Fc binding domain binds a target cell. In this embodiment, the antibody comprises specificity to a target cell antigen. The target cell antigen may include the same target cell antigen that the T cell receptor binds or may include a different target cell antigen. The target cell antigen may include any type of ligand that defines the target cell. For example, the target cell antigen may be chosen to recognize a ligand that acts as a cell marker on target cells associated with a particular disease state. Thus examples of cell markers that may act as ligands for the antibody, include those associated with viral, bacterial and parasitic infections, autoimmune disease and cancer cells.

In one embodiment, the target cell antigen includes any tumor associated antigen (TAA) and viral antigen, or any fragment thereof. In this embodiment, the antibody specifically binds to the target cell antigen.

### Vector Comprising the Fc Immune Receptor

The present invention also includes a vector comprising an isolated nucleic acid sequence encoding an Fc immune receptor as described herein. In one aspect, the invention includes a vector comprising an isolated nucleic acid sequence encoding an immune receptor (IR), wherein the isolated nucleic acid sequence comprises a human nucleic acid sequence of a Fc receptor (Fc) binding domain and a nucleic acid sequence of an intracellular domain of a costimulatory molecule.

The nucleic acid can be cloned into any number of different types of vectors. In one embodiment, the vector comprises a plasmid vector, viral vector, retrotransposon (e.g. piggyback, sleeping beauty), site directed insertion vector (e.g. CRISPR, zinc finger nuclease, TALEN), or suicide expression vector, or other known vector in the art. For example, the nucleic acid can be cloned into a vector including, but not limited to a plasmid, a phagemid, a phage derivative, an animal virus, and a cosmid.

Vectors of particular interest include expression vectors, replication vectors, probe generation vectors, and sequencing vectors. The expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Vectors, including those derived from adenoviruses, adeno-associated viruses, herpes viruses, lentiviruses, and retroviruses are suitable tools to achieve long-term gene transfer since they allow long-term, stable integration of a transgene and its propagation in daughter cells. Lentiviral vectors have the added advantage over vectors derived from onco-retroviruses, such as murine leukemia viruses, in that they can transduce non-proliferating cells, such as hepatocytes. They also have the added advantage of resulting in low immunogenicity in the subject into which they are introduced.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

All vectors discussed herein are capable of use with 3rd generation lentiviral vector plasmids, other viral vectors, or RNA approved for use in human cells. In one embodiment, the vector is a viral vector, such as a lentiviral vector. In another embodiment, the vector is an RNA vector.

The production of the Fc immune receptor by the vector can be verified by sequencing. Expression of the full length Fc immune receptor protein may be verified using immunoblot, immunohistochemistry, flow cytometry or other technology well known and available in the art.

The present invention also provides a vector into which the nucleic acid sequence of the present invention is inserted. The expression of natural or synthetic nucleic acids within a vector construct is typically achieved by operably linking a nucleic acid or portions thereof to a promoter, and incorporating the construct into an expression vector. The vector is one generally capable of replication in a mammalian cell, and/or also capable of integration into the cellular genome of the mammal. Typical vectors contain transcription and translation terminators, initiation sequences, and promoters useful for regulation of the expression of the desired nucleic acid sequence.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the elongation factor-1α promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Introduction of Nucleic Acids

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). Nucleic acids can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). Nucleic acids can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. RNA vectors include vectors having a RNA promoter and/other relevant domains for production of a RNA transcript. Viral vectors, and especially retroviral vectors, have become the most widely used method for inserting genes into mammalian, e.g., human cells. Other viral vectors may be derived from lentivirus, poxviruses, herpes simplex virus, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the Fc immune receptor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one embodiment, a nucleic acid nucleic acid encoding an immune receptor is introduced by a method selected from the group consisting of transducing the population of cells, transfecting the population of cells, and electroporating the population of cells. In one embodiment, a population of cells comprises the isolated nucleic acid sequence encoding the immune receptor as described herein.

In one embodiment, the nucleic acids introduced into the cell are RNA. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is an immune receptor.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

The disclosed methods can be applied to the modulation of T cell activity in basic research and therapy, in the fields of cancer, stem cells, acute and chronic infections, and autoimmune diseases, including the assessment of the ability of the modified T cell to kill a target cancer cell.

### Sources of T Cells

In one embodiment, a source of T cells is obtained from a subject. Non-limiting examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, umbilical cord, and tumors. In certain embodiments, any number of T cell lines available in the art, may be used. In certain embodiments, T cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood by lysing the red blood cells and depleting the monocytes, for example, by centrifugation through a PERCOLL™ gradient. Alternatively, T cells can be isolated from umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19 and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In one embodiment, a population of cells comprise the T cells of the present invention. Examples of a population of cells include, but are not limited to, peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

### Hematologic Cancer

Hematological cancer conditions are the types of cancer such as leukemia and malignant lymphoproliferative conditions that affect blood, bone marrow and the lymphatic system. Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

In AML, malignant transformation and uncontrolled proliferation of an abnormally differentiated, long-lived myeloid progenitor cell results in high circulating numbers of immature blood forms and replacement of normal marrow by malignant cells. Symptoms include fatigue, pallor, easy bruising and bleeding, fever, and infection; symptoms of leukemic infiltration are present in only about 5% of patients (often as skin manifestations). Examination of peripheral blood smear and bone marrow is diagnostic. Existing treatment includes induction chemotherapy to achieve remission and post-remission chemotherapy (with or without stem cell transplantation) to avoid relapse.

AML has a number of subtypes that are distinguished from each other by morphology, immunophenotype, and cytochemistry. Five classes are described, based on predominant cell type, including myeloid, myeloid-monocytic, monocytic, erythroid, and megakaryocytic.

Remission induction rates range from 50 to 85%. Long-term disease-free survival reportedly occurs in 20 to 40% of patients and increases to 40 to 50% in younger patients treated with stem cell transplantation.

Prognostic factors help determine treatment protocol and intensity; patients with strongly negative prognostic features are usually given more intense forms of therapy, because the potential benefits are thought to justify the increased treatment toxicity. The most important prognostic factor is the leukemia cell karyotype; favorable karyotypes include t(15;17), t(8;21), and inv16 (p13;q22). Negative factors include increasing age, a preceding myelodysplastic phase, secondary leukemia, high WBC count, and absence of Auer rods.

Initial therapy attempts to induce remission and differs most from ALL in that AML responds to fewer drugs. The basic induction regimen includes cytarabine by continuous IV infusion or high doses for 5 to 7 days; daunorubicin or idarubicin is given IV for 3 days during this time. Some regimens include 6-thioguanine, etoposide, vincristine, and prednisone, but their contribution is unclear. Treatment usually results in significant myelosuppression, with infection or bleeding; there is significant latency before marrow recovery. During this time, meticulous preventive and supportive care is vital.

The present invention provides compositions and methods for treating cancer. In one aspect, the cancer is a hematologic cancer including but is not limited to leukemias (such as acute myelogenous leukemia, chronic myelogenous leukemia, acute lymphoid leukemia, chronic lymphoid leukemia and myelodysplastic syndrome) and malignant lymphoproliferative conditions, including lymphomas (such as multiple myeloma, non-Hodgkin's lymphoma, Burkitt's lymphoma, and small cell- and large cell-follicular lymphoma).

Moreover, it is useful in the context of the present invention that the T cell has limited toxicity toward healthy cells. By targeting the tumor or diseased cells, the T cell manifests no substantial negative biological effects, anti-tumor effects, or substantial negative physiological symptoms toward a healthy cell, non-tumor cell, non-diseased cell, non-target cell or population of such cells either in vitro or in vivo.

### Therapy

The modified cells described herein may be included in a composition for therapy. In one aspect, the composition comprises the modified T cell comprising the immune receptor described herein. In another aspect, the composition comprises the modified cell further comprising an antibody described herein. The composition may include a pharmaceutical composition and further include a pharmaceutically acceptable carrier. A therapeutically effective amount of the pharmaceutical composition comprising the modified cells may be administered.

The modified T cells generated as described herein possess T cell function. Further, the modified T cells can be administered to an animal, preferably a mammal, even more preferably a human, to suppress an immune reaction, such as those common to autoimmune diseases such as diabetes, psoriasis, rheumatoid arthritis, multiple sclerosis, GVHD, enhancing allograft tolerance induction, transplant rejection, and the like. In addition, the cells of the present invention can be used for the treatment of any condition in which a diminished or otherwise inhibited immune response, especially a cell-mediated immune response, is desirable to treat or alleviate the disease. In one aspect, the invention includes treating a condition, such as a disease or condition associated with resistance to an antibody-mediated therapy, in a subject, comprising administering to the subject a therapeutically effective amount of a pharmaceutical composition comprising a population of modified cells.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

The cells of the invention to be administered may be autologous, allogeneic or xenogeneic with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In another embodiment, the present invention provides methods for treating a disease or condition associated with resistance to an antibody-mediated therapy, the methods comprising contacting the Fc-expressing cancer cell population with an FcIR cell of the invention that binds to the Fc-expressing cell. In certain embodiments, the FcIR cell of the invention reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model for myeloid leukemia or another cancer associated with Fc-expressing cells relative to a negative control. In one aspect, the subject is a human.

The present invention also provides methods for preventing, treating and/or managing a disorder associated with Fc-expressing cells (e.g., a hematologic cancer), the methods comprising administering to a subject in need an FcIR cell of the invention that binds to the Fc-expressing cell. In one aspect, the subject is a human. Non-limiting examples of disorders associated with Fc-expressing cells include autoimmune disorders (such as lupus), inflammatory disorders (such as allergies and asthma) and cancers (such as hematological cancers).

The present invention also provides methods for preventing, treating and/or managing a disease associated with antibody resistance, the methods comprising administering to a subject in need an FcIR of the invention that binds to the Fc-expressing cell. In one aspect, the subject is a human. Non-limiting examples of diseases associated with Fc-expressing cells include Acute Myeloid Leukemia (AML), myelodysplasia, B-cell Acute Lymphoid Leukemia, T-cell Acute Lymphoid Leukemia, hairy cell leukemia, blastic plasmacytoid dendritic cell neoplasm, chronic myeloid leukemia, Hodgkin's lymphoma, and the like.

The present invention provides methods for preventing relapse of cancer associated with antibody resistance, the methods comprising administering to a subject in need thereof a FcIR cell of the invention that binds to the Fc-expressing cell. In one aspect, the invention includes a method of treating a tumor in a mammal, the method comprising administering to the subject an effective amount of a genetically modified cell comprising an immune receptor (IR), wherein the immune receptor comprises a Fc receptor (Fc) binding domain and an intracellular domain of a costimulatory molecule. In one embodiment, the method comprises administering to the subject in need thereof an effective amount of a FcIR cell of the invention that binds to the Fc-expressing cell in combination with an effective amount of another therapy. In another embodiment, the method comprises administering an antibody specific for a target cell prior to administering the effective amount of the modified T cell. In yet another embodiment, the method comprises binding the modified T cell to an antibody with specificity for a target cell.

In one aspect, the invention includes a method of treating a mammal having a disease, disorder or condition associated with antibody resistance, the method comprising administering to the subject an effective amount of a genetically modified cell comprising an isolated nucleic acid sequence encoding an immune receptor (IR), wherein the isolated nucleic acid sequence comprises a human nucleic acid sequence of a Fc receptor (Fc) binding domain and a nucleic acid sequence of an intracellular domain of a costimulatory molecule.

In another aspect, the invention includes a method for treating antibody resistance or increasing response to antibody therapy in a subject, the method comprising: administering to the subject an effective amount of a genetically modified T cell comprising an isolated nucleic acid sequence encoding an immune receptor (IR), wherein the isolated nucleic acid sequence comprises a human nucleic acid sequence of a Fc receptor (Fc) binding domain and a nucleic acid sequence of an intracellular domain of a costimulatory molecule, thereby treating antibody resistance or increasing response to antibody therapy in the subject.

In one aspect, the invention includes a method of treating a disease or condition associated with resistance to an antibody-mediated therapy in a subject comprising administering to the subject a therapeutically effective amount of a pharmaceutical composition comprising the modified T cell described herein. In another aspect, the invention includes a method of treating a condition in a subject, comprising administering to the subject a therapeutically effective amount of a pharmaceutical composition comprising the modified T cell described herein. In another aspect, the invention includes a method for stimulating a T cell-mediated immune response to a target cell or tissue in a subject comprising administering to a subject a therapeutically effective amount of a pharmaceutical composition comprising the modified T cell described herein. In yet another aspect, the invention includes use of the modified T cell described herein in the manufacture of a medicament for the treatment of an immune response in a subject in need thereof. In these embodiments, the T cell comprises an immune receptor (IR) comprising a Fc receptor (Fc) binding domain and an intracellular domain of a costimulatory molecule. Another embodiment includes the T cell further comprising an antibody.

Another aspect of the invention includes a method for overcoming resistance to an antibody-mediated therapy in a subject, the method comprising administering to the subject an effective amount of a modified T cell comprising an immune receptor (IR), wherein the immune receptor comprises a Fc receptor (Fc) binding domain and an intracellular domain of a costimulatory molecule, thereby overcoming resistance to the antibody-mediated therapy in the subject.

The methods of the present invention are particularly useful for humans, but may also be practiced on veterinary subjects. An “individual,” “subject,” “patient” or “host” referred to herein is a vertebrate, preferably a mammal. More preferably, such individual is a human and the culture-expanded cells are human, although animals, including animal models for human disease states, are also included in this invention and therapeutic treatments of such animals are contemplated herein. Such animal models can be used to test and adjust the compositions and methods of this invention, if desired. Certain models involve injecting in-bred animals with established cell populations. Also useful are chimeric animal models, described in U.S. Pat. Nos. 5,663,481, 5,602,305 and 5,476,993; EP application 379,554; and International Appl. WO 91/01760. Non-human mammals include, but are not limited to, veterinary or farm animals, sport animals, and pets. Accordingly, as opposed to animal models, such animals may be undergoing selected therapeutic treatments.

Based upon the disclosure provided herein, helper T cells can be obtained from any source, for example, from the tissue donor, the transplant recipient or an otherwise unrelated source (a different individual or species altogether). The helper T cells may be autologous with respect to the T cells (obtained from the same host) or allogeneic with respect to the T cells. In the case where the helper T cells are allogeneic, the helper T cells may be autologous with respect to the transplant to which the T cells are responding to, or the helper T cells may be obtained from a mammal that is allogeneic with respect to both the source of the T cells and the source of the transplant to which the T cells are responding to. In addition, the helper T cells may be xenogeneic to the T cells (obtained from an animal of a different species), for example rat helper T cells may be used to suppress activation and proliferation of human T cells.

Another embodiment of the present invention encompasses the route of administering helper T cells to the recipient of the transplant. The helper T cells can be administered by a route which is suitable for the placement of the transplant, i.e. a biocompatible lattice or a donor tissue, organ or cell, nucleic acid or protein, to be transplanted. The helper T cells can be administered systemically, i.e., parenterally, by intravenous injection or can be targeted to a particular tissue or organ, such as bone marrow. The helper T cells can be administered via a subcutaneous implantation of cells or by injection of the cells into connective tissue, for example, muscle.

The helper T cells can be suspended in an appropriate diluent, at a concentration of about 5×10⁶ cells/ml. Suitable excipients for injection solutions are those that are biologically and physiologically compatible with the helper T cells and with the recipient, such as buffered saline solution or other suitable excipients. The composition for administration can be formulated, produced and stored according to standard methods complying with proper sterility and stability.

The dosage of the helper T cells varies within wide limits and may be adjusted to the mammal requirements in each particular case. The number of cells used depends on the weight and condition of the recipient, the number and/or frequency of administrations, and other variables known to those of skill in the art.

Between about 10⁵ and about 10¹³ helper T cells per 100 kg body weight can be administered to the mammal. In some embodiments, between about 1.5×10⁶ and about 1.5×10¹² cells are administered per 100 kg body weight. In some embodiments, between about 1×10⁹ and about 5×10¹¹ cells are administered per 100 kg body weight. In some embodiments, between about 4×10⁹ and about 2×10¹¹ cells are administered per 100 kg body weight. In some embodiments, between about 5×10⁸ cells and about 1×10¹⁰ cells are administered per 100 kg body weight.

### Pharmaceutical Compositions

Pharmaceutical compositions of the present invention may comprise a modified cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-immune response effective amount”, “an immune response-inhibiting effective amount”, or “therapeutic amount” is indicated, the precise amount of the compositions of the present invention to be administered can be determined by a physician with consideration of individual differences in age, weight, immune response, and condition of the patient (subject). It can generally be stated that a pharmaceutical composition comprising the modified T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated T cells. This process can be carried out multiple times every few weeks. In certain embodiments, T cells can be activated from blood draws of from 10 ml to 400 ml. In certain embodiments, T cells are activated from blood draws of 20 ml, 30 ml, 40 ml, 50 ml, 60 ml, 70 ml, 80 ml, 90 ml, or 100 ml. While not wishing to be bound by theory, using this multiple blood draw/multiple reinfusion protocol, may select out certain populations of T cells.

In certain embodiments of the present invention, T cells are modified using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

It should be understood that the methods described herein may be carried out in a number of ways and with various modifications and permutations thereof that are well known in the art. It may also be appreciated that any theories set forth as to modes of action or interactions between cell types should not be construed as limiting this invention in any manner, but are presented such that the methods of the invention can be more fully understood.

The following examples further illustrate aspects of the present invention. However, they are in no way a limitation of the teachings or disclosure of the present invention as set forth herein.

These methods described herein are by no means all-inclusive, and further methods to suit the specific application will be apparent to the ordinary skilled artisan. Moreover, the effective amount of the compositions can be further approximated through analogy to compounds known to exert the desired effect.

The materials and methods employed in these experiments are now described.

Fc Ig Binding Immune Receptor Construction (FcIR).

Human CD64, DNA sequence was amplified from primary human monocytes using primers. After amplification and the insertion of 3′-Bam-H1 and 5′-Nhe-1 restriction sites, PCR product was digested with Bam-HI and NheI enzymes and ligated into pELNS, a third generation self-inactivating lentiviral expression vector, containing human CD3z or CD28-CD3z signaling endodomains, under an EF-1a promoter. The resulting constructs were designated pELNS CD64 FcIR-zeta and pELNS Cd64 FcIR-28z, respectively.

Recombinant Lentivirus Production.

High-titer replication-defective lentiviral vectors were produced and concentrated. Briefly, 293T cells were transfected with pVSV-G (VSV glycoprotein expression plasmid), pRSV.REV (Rev expression plasmid), pMDLg/p.RRE (Gag/Pol expression plasmid), and pELNS transfer plasmid using Lipofectamine 2000 (Invitrogen). The viral supernatant was harvested at 24 and 48 h post-transfection.

Lymphocytes.

Primary human CD4+ and CD8+ T-cells isolated from healthy volunteer donors were purchased from the Human Immunology Core at University of Pennsylvania, activated and transduced with lentiviral vectors. Human recombinant interleukin-2 (IL-2; Novartis) was added every other day to 50 IU/ml final concentration and a 0.5-1×10⁶ cells/ml cell density was maintained. Rested engineered T cells were adjusted for identical transgene expression prior to functional assays.

Cell Lines.

Lentivirus packaging was performed in the immortalized normal fetal renal 293T cell line purchased from ATCC. Human cell lines used in immune based assays include CD20 positive cell lines Ramos and Daudi. 293T cells and tumor cell lines were maintained in RPMI-1640 (Invitrogen) supplemented with 10% (v/v) heat-inactivated FBS, 2 mM L-glutamine, and 100 μg/mL penicillin and 100 U/mL streptomycin. All cell lines were purchased from ATCC.

Flow Cytometric Analysis.

APC-Cy7 Mouse Anti-Human-CD3; FITC-anti-human-CD4; APC-anti-human-CD8; PE-human-CD45; APC-human-CD69 antibodies were purchased from (Biolegend). FSHR expression was detected using clone6266717 (R&D Systems). T-cell transduction was measured by GFP transgene expression. 7AAD (Biolegend) was used to assess viability. For in vivo T-cell quantification, 50 μL blood was obtained from mice via retro-orbital bleeding and labeled for human CD45, CD3, and CD8. Cell numbers were quantified using BD TruCount tubes per manufacturer's instructions. Flow cytometry data were analyzed using FlowJo software. Tumor cell surface expression of HER2 was detected by trastuzumab antibody (Herceptin, Genentech, San Francisco, Calif.), CD20 by biotinylated Rituximab (rituxan; Genentech, South San Francisco, Calif., USA.) followed by incubation with Strepavidin-APC. FcIR expression was detected by anti-CD64, CD32 and CD16-APC AB (BioLegend) Isotype matched control Abs were used in all analyses. Flow cytometric data were analyzed by FlowJo software.

Antibody Arming of T Cells.

The antibody-binding capacity of FcIR T-cells or a control GFP transduced T-cells was determined by flow analysis. T-cells were incubated with IgG1 mouse, trastuzumab, retixumab, IgG2a for 30 min in RT. After washing twice with PBS, cells were incubated with goat anti-muman IgG conjugated to APC, or Stre-AV FITC, APC for 15 min at room temperature. Cell staining analysis was performed by flow cytometry.

Cytokine Release Assays.

Cytokine release assays were performed by co-culture of 1×10⁵ FcIR⁺T cells with immobilized antibodies; IgG1 (100 ng/ml). For co-culture experiments against tumor cells, 1×10⁵ target cells were labeled with TAA specific Abs at 100 ng/10⁶ cells for 30 min at 4° C., per well in triplicate in 96-well round bottom plates, T-cells were added into the culture at the E:T 1:1 ratio, in a final volume of 200 ul of T cell media. After 16 h, co-culture supernatants were assayed for presence of IFNγ using an ELISA Kit, according to manufacturer's instructions (Biolegend). Values represent the mean of triplicate wells. IFNγ, IL-2, IL-4, TNFa and MIP-1a cytokines were measured by flow cytometry using Cytokine Bead Array, according to manufacturer's instructions (BD Biosciences).

Cytotoxicity Assays.

⁵¹Cr release assays were performed as described. Target cells were labeled with Abs at 100 ng per 10⁶ cells for 30 min at 37° C. in PBS/2% FBS. Next, antibody labeled cells were labeled with 100 uCi 100 mCi ⁵¹Cr at 37° C. for 1.5 hours. Target cells were washed three times in PBS, resuspended in CM at 10⁵ viable cells/mL and 100 uL added per well of a 96-well V-bottom plate. Effector cells were washed twice in CM and added to wells at the given ratios. Plates were quickly centrifuged to settle cells, and incubated at 37° C. in a 5% CO₂ incubator for 4 hours. The supernatants were harvested, transferred to a lumar-plate (Packard) and counted using a 1450 Microbeta Liquid Scintillation Counter (Perkin-Elmer). Spontaneous ⁵¹Cr release was evaluated in target cells incubated with medium alone. Maximal ⁵¹Cr release was measured in target cells incubated with SDS at a final concentration of 2% (v/v). Percent specific lysis was calculated as (experimental−spontaneous lysis/maximal−spontaneous lysis) times 100.

In Vivo Studies.

NOD/SCID/γ-chain−/− (NSG) mice were bred, treated, and maintained under pathogen-free conditions in-house under University of Pennsylvania IACUC-approved protocols. Six to twelve week old female mice were purchased from the University of Pennsylvania Stem Cell and Xenograft Core and 5×10⁶ CaOV3-fLuc tumor cells were inoculated subcutaneously (5 mice/group). Twenty and 25 days later, mice were injected intraperitoneally with 6×10⁶ T-cells. Tumor growth was assessed by weekly caliper measurements. Tumor volume was calculated using the following formula: V=½(length×width²), where length is greatest longitudinal diameter and width is greatest transverse diameter.

Statistical Analysis.

Student's t-test was used to evaluate differences in T-cells specific cytolysis and cytokine secretion. GraphPad Prism 4.0 (GraphPad Software) was used for the statistical calculations. P<0.05 was considered significant.

The results of the experiments are now described Reports of poor antitumor effects of Ab-therapy in cancer led to the hypothesis that the development of potent effector cells with the capacity to bind tumor-bound monoclonal antibody (mAb) mediate strong antibody-directed cellular cytotoxicity (ADCC) would markedly improve the efficacy of mAb-targeted therapy.

There are multiple causes why antibody treatment of patients with malignant tumors may not achieve a successful therapeutic effect. These account for: the heterogeneity of target antigen expression in the tumor, intratumoral microenvironment, as well as immune escape through ineffective FcγR binding. Therefore, at present, the concept of potentiating the cytotoxicity induced by anticancer mAbs has been the subject of efforts to enhance the downstream cytolytic effector cells/mechanism of ADCC. Notably, clinical results have shown that patients harboring an FcγRIIIA polymorphism, FcγR-IIIA-157V, have NK cells with a higher affinity for IgG1, have favorable response rates to cetuximab treatment in colorectal cancer and rituximab in follicular lymphoma. However, this association was not observed in HER2/neu breast cancer patients treated with trastuzumab.

On the basis of these results, it was hypothesized that antibody based immunotherapy could be enhanced by engineering a potent effector cell, with tumor specificity directed by antigen bound antibody. The recruitment of T cells engineered to express Fcγ binding receptor linked to intracellular T cell signaling domains appeared well suited in this regard, because several lines of evidence suggested that T cell transfer expressing chimeric immune receptor showed a superior cytolytic activity and possibly overcame inhibitory molecules. Moreover, engineered T cells can be amplified and expanded in vitro to high numbers, tuned for stronger ADCC activity boosted by providing a co-stimulation.

In order to expand applications for T cell-based immunotherapy and enhance ADCC, a novel effector T-cell engineered was engineered to express Fc-binding immune receptor (FcIR) containing a human Fc-receptor of low FcγRIIIA (CD16), intermediate FcγRIIA (CD32), or high affinity FcγRI (CD64) molecules fused to a co-stimulatory signaling domain in order to enable cytotoxic T-cells to mediate strong mAb-directed cytotoxicity against antigen-expressing tumor cells.

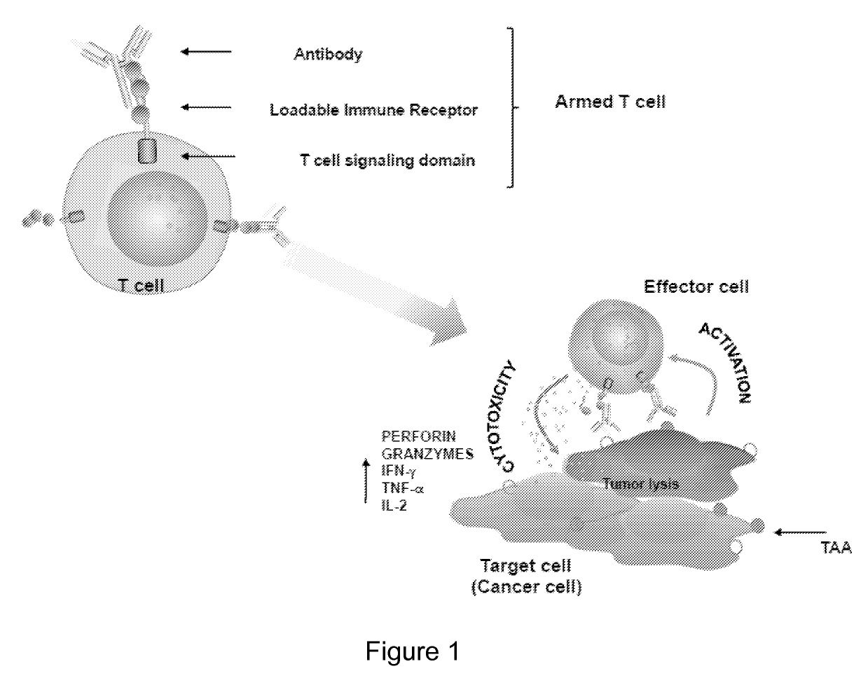

There were concerns that tumor cells were evading antibody-dependent cellular cytotoxicity (ADCC) and other immune functions due to tumor factors, such as poor penetration of effector cells into solid tumor tissue, complement-regulatory proteins present on the tumor cells, altered signaling and soluble antigens, and host factors, such as heterogeneity in ADCC according to FcγRIIIa (II) functional polymorphism, inactivation of effector cells in patients (downregulation of ζ chain, NKG2D, NKp30, NKp44, NKp46 in NK cells), and suppression of effector cells by chemotherapy. A novel platform was developed for antibody directed cellular cytotoxicity that utilized non-immunogenic loadable, clinical grade antibodies for strong antitumoral effects in vivo. The platform employed an immune receptor, see FIG. 1, that included an extracellular antibody bound to a loadable immune receptor and an internal T cell signaling domain. The immune receptor was bound to antibody, such as those listed in Table 1, and was exposed on the surface of the T cell, thus, arming the T cell. Upon specific binding by the antibody to its antigen on a target cell, activation of the T cell effector functions occurred, i.e. the release of perforin, granzymes, IFN-γ, TNF-α, and IL-2.

TABLE 1
Antibodies used in antibody-dependent cellular cytotoxicity (ADCC).

| | | | | | Initial |
|---|---|---|---|---|---|
| | | | | | assessment |
| | | | | | of effector |
| Generic name | | | Approved | Proposed mechanisms | function |
| (trade name) | Format | Targets | indications | of action* | potential^{\|\|} |
| Alemtuzumab | Humanized | CD52 | CLL | Induction of ADCC | High |
| (Campath; | IgG1κ | | | | |
| Cetuximab | Chimeric | EGFR | Metastatic | Inhibition of EGFR | High |
| | (murine/human) | | colorectal | signalling; induction of | |
| | IgG1κ | | cancer, and head | apoptosis and ADCC; | |
| | | | and neck cancer | sensitization of cells to | |
| | | | | chemotherapy and | |
| | | | | radiotherapy | |
| Ofatumumab | Human IgG1κ | CD20 | CLL | Induction of CDC and | High |
| Genmab | | | | ADCC | |
| Rituximab | Chimeric | CD20 | Non-Hodgkin's | Induction of apoptosis, | High |
| (Rituxan) | (murine/human) | | lymphoma, RA | ADCC and CDC; | |
| | IgG1κ | | and CLL | sensitization of cells to | |
| | | | | chemotherapy | |
| Trastuzumab | Humanized | HER2 | HER2- | Inhibition of HER2 | High |
| (Herceptin;) | IgG1κ | | overexpressing | signaling; induction of | |
| | | | breast cancer | ADCC; sensitization of | |
| | | | | cells to chemotherapy | |

### Construction of a High Affinity Fc Binding Immune Receptor (FcIR)

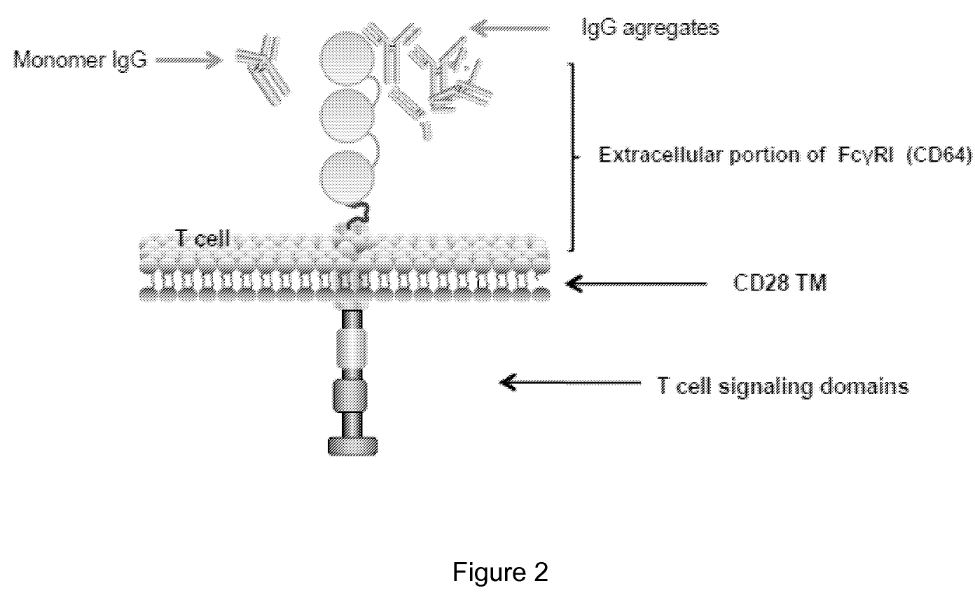

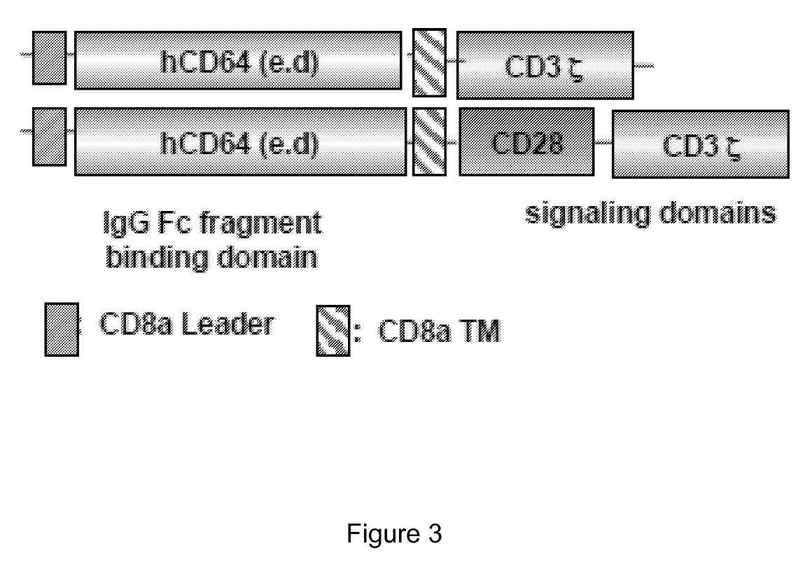

The advantages of antibody and adoptive T cell transfer therapies are merged in an antibody Fc binding immunoreceptor, comprising extracellularly expressed CD64 molecule (FcgRIa) linked to intracellular T cell signaling domains via the transmembrane domain of CD8 followed by a CD3z signaling moiety alone (FcIR-z). Furthermore, since the importance of a second co-stimulatory signal is well established, a second-generation FcIR that includes a fusion of both chimeric CD28 and CD3-z intracellular domains (FcIR-28z) is shown (FIGS. 2 and 3).

The lentiviral vector system was used for primary human T cell transduction. pELNS based vectors encoding for FcIR-z and FcIR-28z as well, as control GFP vector resulted in a median transgene transduction efficiency: median expression in CD3+ cells for chimeric receptor surface expression as assessed by anti-CD64 antibody.

Antibody-binding capacity of primary human T cells expressing CD64 FcIR-28z was assessed 5 days following lentiviral transduction of T lymphocytes expressing CD64-FcIR 28z coated with mouse FITC conjugated IgG2a antibody. Notably, only FcIR expressing T cells, detectable by anti-CD64-APC conjugated antibody, captured IgG2a-FITC antibody on their surface as analyzed by flow cytometry. Importantly, control, untransduced T cells were not coated with the antibody.

The binding specificity of CD64-FcIR-28z was assessed. According to the literature, human CD64 binds mouse IgG2a, but not IgG1 isotype. Therefore, transduced primary and control T cells with increasing concentrations of IgG2a and IgG1 antibodies were analyzed for their presence on the cell surface by FACS. CD64-FcIR-28z bound specifically to IgG2a isotype, in a dose dependent manner, but did not bind IgG1a antibody. Captured IgG2a was also detected at antibody concentrations as low as 10 ng per 1 million cells. The data shows a linear correlation between the concentration of IgG2a antibody used and specific mean fluorescence intensity. Control, untransduced T cells did not bind IgG2a antibody.

### Binding of Tumor Specific Antibody Induces Antigen Dependent Activation of FcIR T-Cell

Antibody binding to the CD64FcIR-28z was redirected FcIRs against target tumor cells. FcIR-28z T cells were incubated with anti-EpCAM IgG2a antibody, at the indicated concentrations and mixed at a ratio 1:1 with an EpCAM positive ovarian cancer cell line, A1847 or cultured without the targets for 16 hrs. Anti-EpCAM IgG2a antibody bound to CD64-FcIR 28z and induced activation of FcIRs only in the presence of EpCAM positive target cell lines, as assessed by production of IFNγ. Whereas no IFNγ cytokine production was observed when CD64FcIR T cells were armed with anti-EpCAM antibody but were not exposed to target antigen.

Moreover, IFNγ analysis did not show a significant IFNγ production by FcIR-T cells when armed with antigen specific antibody, or control IgG2a isotope. T lymphocytes transduced with CD64FcIR did not produce IFNγ when cultured with the tumor target cell line, A1847, in the presence of IgG2a isotype control antibody.

In addition, the specific immunoreactivity of FcIR T cells was analyzed when co-cultured with “painted targets” or targets coated with antibody. FcIR transduced T lymphocytes markedly increased IFNγ production when cultured with anti EpCAM IgG2a painted, human ovarian cancer cells, A1847, whereas no changes in immunoreactivity were detected when cultured with targets painted with anti-FRα IgG1 antibody, or in the absence of an antibody.

In addition to IFNγ production, CD64-FcIR-28z T cells produced high levels of Th1 type cytokines, including IL2, TNFalpha, and MIP1 alpha.

### Primary Human T-Cells Engineered to Express FcIR Mediate Potent ADCC In Vitro

CD64FcIR T cells released high levels of cytokines when redirected against target cells with specific antibodies. Therefore, FcIRs were hypothesized to specifically lyse target tumor cells in the presence of TAA specific antibodies. In in vitro cytotoxcicity assays, CD64FcIR T lymphocytes were cytotoxic against the EpCAM positive ovarian cancer cell line, A1847, in the presence of anti-EpCAM IgG2a specific antibody. They showed approximately 30% of the target cells were specifically lysed following co-culture at a 1:30 E:T ratio at an antibody concentration as low as 10 ng per 1 million cells. Notably, target cell killing was not observed when CD64FcIR were armed with control IgG2a antibody, which lacks antigen specificity. Control, primary human T cells also did not lyse tumor target cells in any of the tested conditions. By prolonging the culture, cytotoxicity exceeded 80% at even lower E:T ratios in the presence of anti EpCAM armed FcIRs.

In further cytoxicity experiments with EpCAm positive, A1847, ovarian cancer cell line, cytotoxicity was achieved against painted targets. FcIR T cells were cultured with A1847, ovarian cancer cells, labeled with anti EpCAM antibody at the indicated concentrations. Only CD64FcIR engineered T cells were able to mediate specific cytotoxicity in the presence of painted targets. Importantly, FcIR T cells armed with anti-EpCAM antibody were not cytotoxic against EpCAM negative target cells.

Other immunotherapeutic antibodies were tested to see if they elicited similar cytotoxic activity as CD64FcIR engineered T cells. Thus, CD64FcIR T cells were tested against a panel of tumor cell lines expressing EpCAM and different levels of HER2 antigen including breast cancer cell lines, MDA MB-453, -361 and MCF7, as well as ovarian cancer cells, A1847. Breast cancer cell line, MDA MB-468, was HER2 negative and was used as an antigen specificity control. FcIR T cells were redirected against EpCAM antigen or HER2 via arming with anti-EpCAM IGg2a Ab and anti-HER2, trastuzumab mAB.

FcIRs were also armed with rituximab antibody, specific for CD20, and used in the assays as an antigen specificity control. After 16 hrs following co-culture with target cancer cell lines, FcIRs produced high levels of IFNγ only in the presence of antigen specific antibodies. Importantly, even low levels of targeted antigen, HER2, expressed on MCF7 and A1847 cell lines triggered antigen specific cytokine release. Subsequently, different immunotherapeutic antibodies were thought to trigger comparable cytotoxicities against tumor cells expressing the specific antigen. Therefore, the cytotoxicity of FcIR T cells was tested against tumor cells expressing HER2 and EpCAM (breast cancer cell line MDA-MB 453, 361 and ovarian cancer cell line A1847). In order to redirect antigen specificity against HER2, trastuzumab antibody was used, to redirect against EpCAM, and anti-EpCAM IgG2a antibody was used and to redirect against CD20, rituxan was used.

Human CD64 Fc-binding immune receptor including an extracellular portion of human CD64 was fused to CD28 transmembrane and T cell signaling domains (FIGS. 2 and 3). CD64Fc immune receptor T cells were redirected against tumor-associated antigens (TAAs) via arming the T cells with TAA specific antibodies. CD64 Fc immune receptor provided co-stimulatory signals which enhanced T cells persistence and effector function. The immune receptor displayed extensive flexibility for targeting different TAAs, while providing co-stimulatory signals within the T cell to activate effector functions.

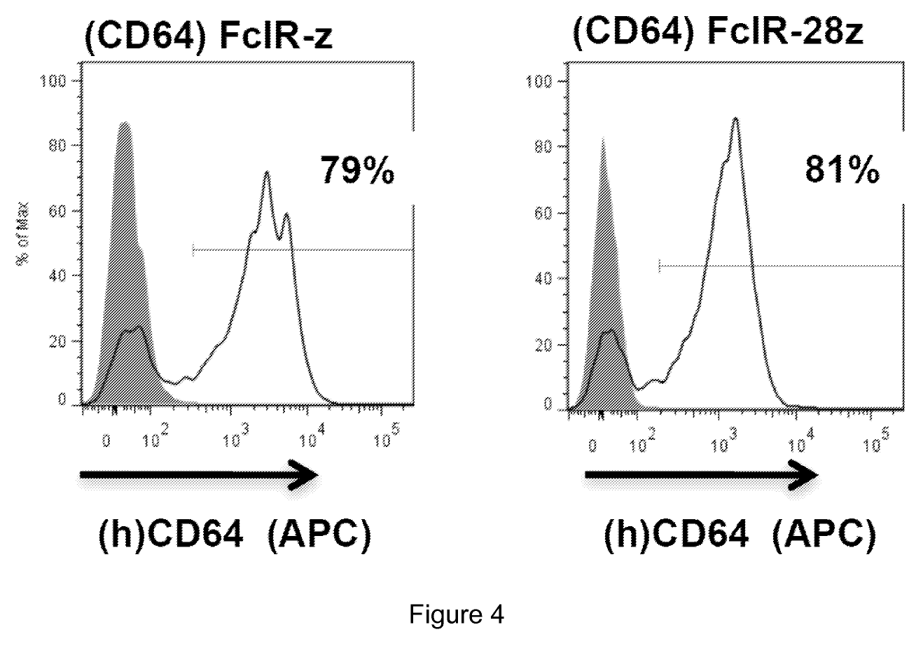

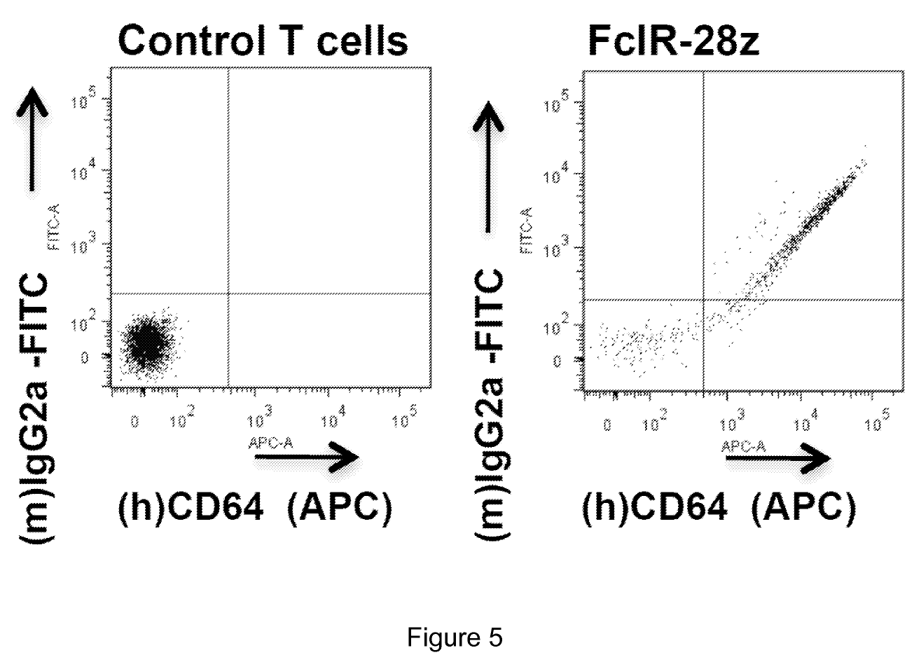

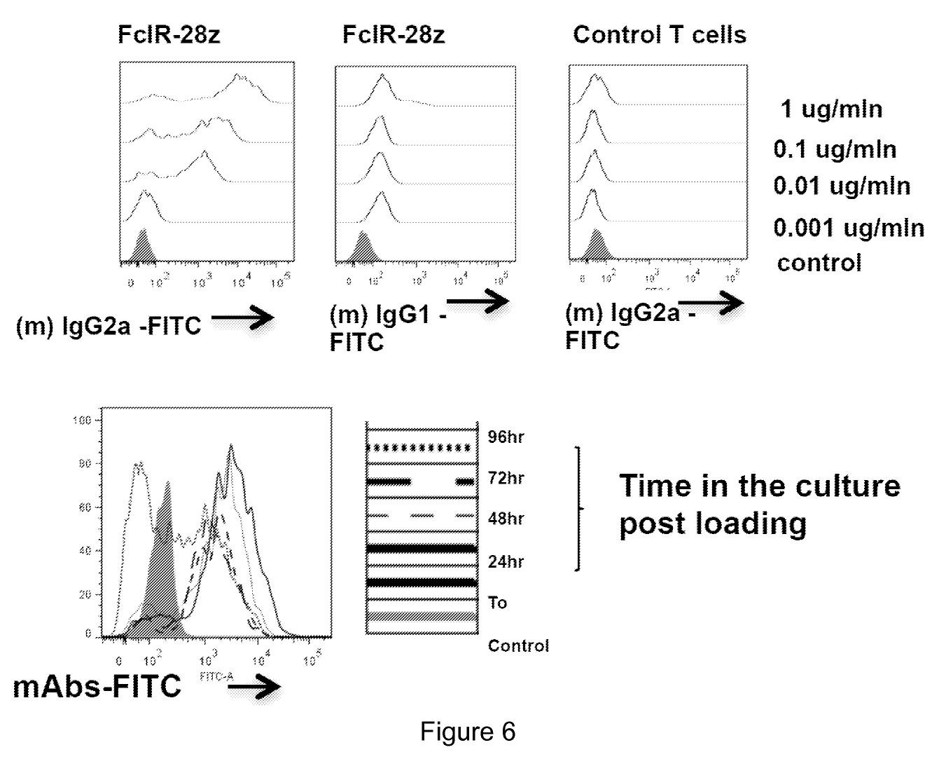

Fc immune receptors constructs with CD3zeta or CD28-CD3zeta intracellular signaling domains (FIG. 3) expressed in primary human T cells (FIG. 4). CD64 is the high-affinity IgG Fc receptor. When exposed to mouse IgG2a, the Fc immune receptors bound the antibodies (FIG. 5). The Fc immune receptor expressing T cells trapped the antigen specific antibodies (upper diagrams of FIG. 6) and retained them for extended periods of time (lower diagram of FIG. 6).

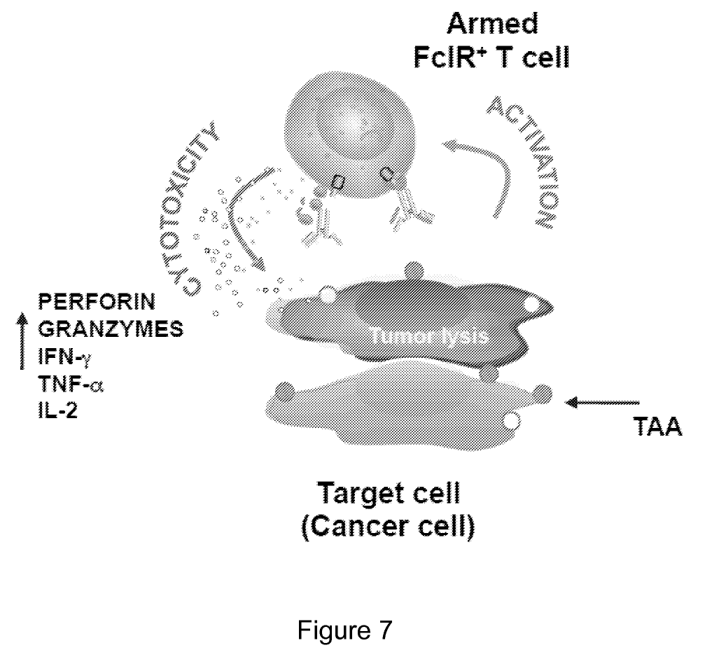

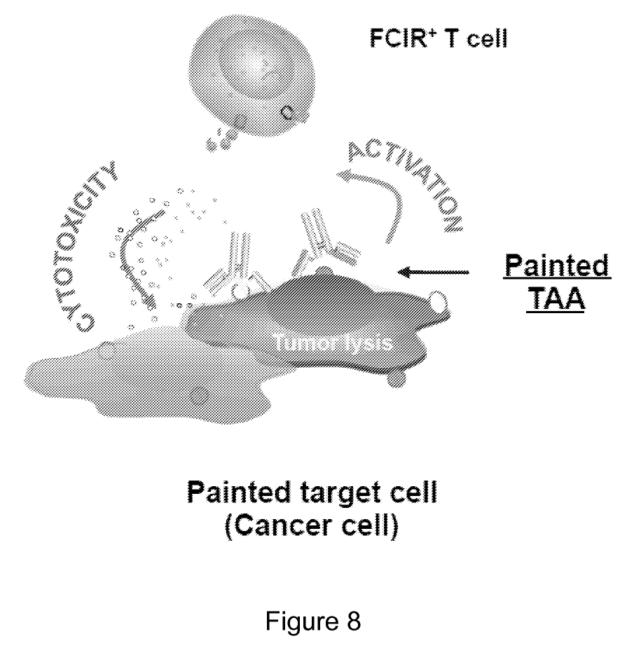

Two potential mechanisms for taking advantage of Fc immune receptor T cell mediated cytoxicity are shown in FIGS. 7 and 8. FIG. 7 illustrates administration of T cells expressing Fc immune receptors with specific antibody bound to the Fc immune receptors. Specific binding of the antibody to the target antigens on a target cell induced cytotoxicity. FIG. 8 illustrates pre-administration of antibody to specifically bind to antigen on target cells followed by administration of T cells expressing Fc immune receptors. The Fc immune receptor on the T cells specifically bound the target cell-bound antibody and induced cytotoxicity. Arming CD64 Fc immune receptor T cells with an antigen specific antibody allowed for redirection against a chosen antigen specificity. CD64 Fc immune receptor T cells were redirected against EpCAM (tumor-associated antigen) by loading the immune receptor with anti-EpCAM IgG2a antibody. MOV-18 IgG1 type antibody was used as a control for isotope specificity. Untransduced T cells did not retain immunoglobulin on their cell surface

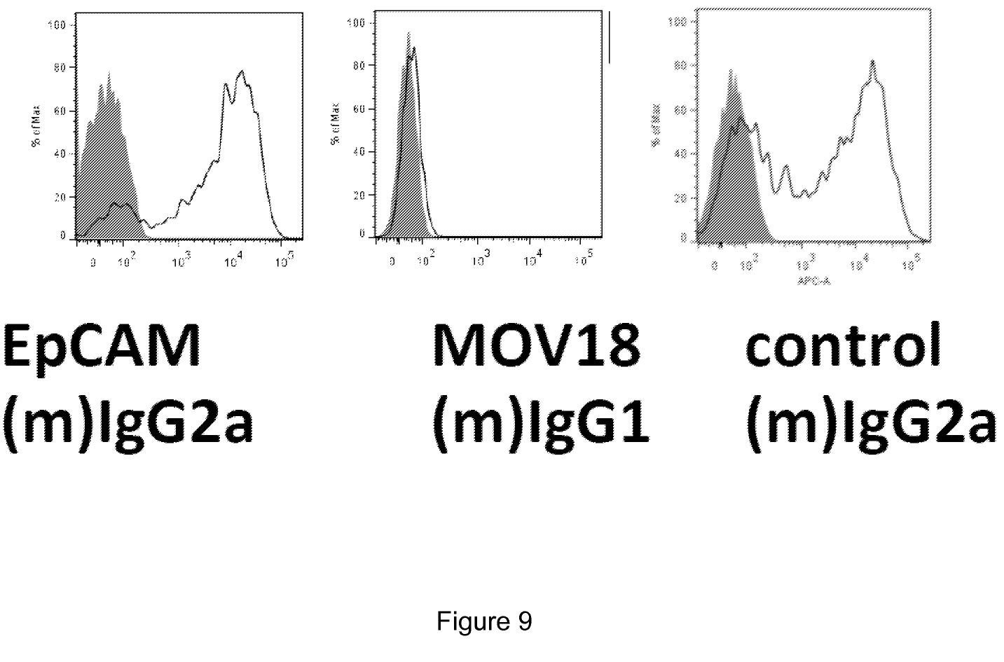

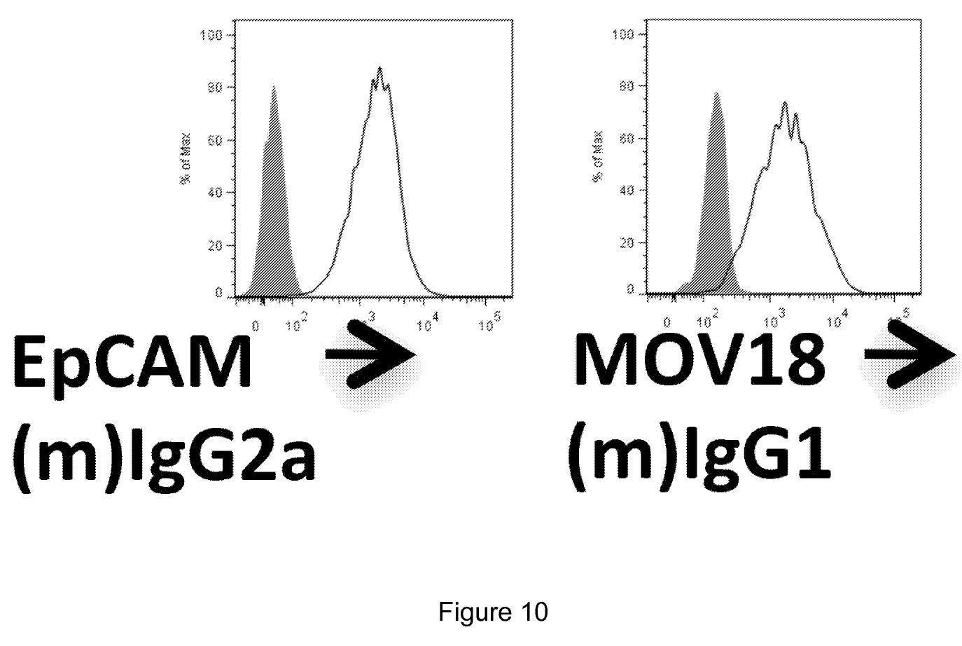

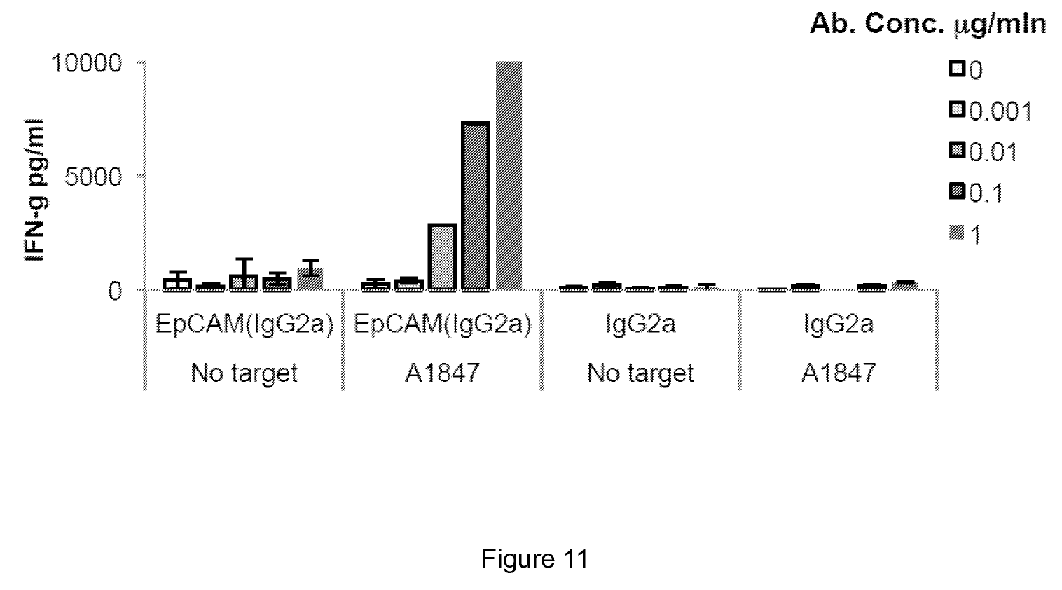

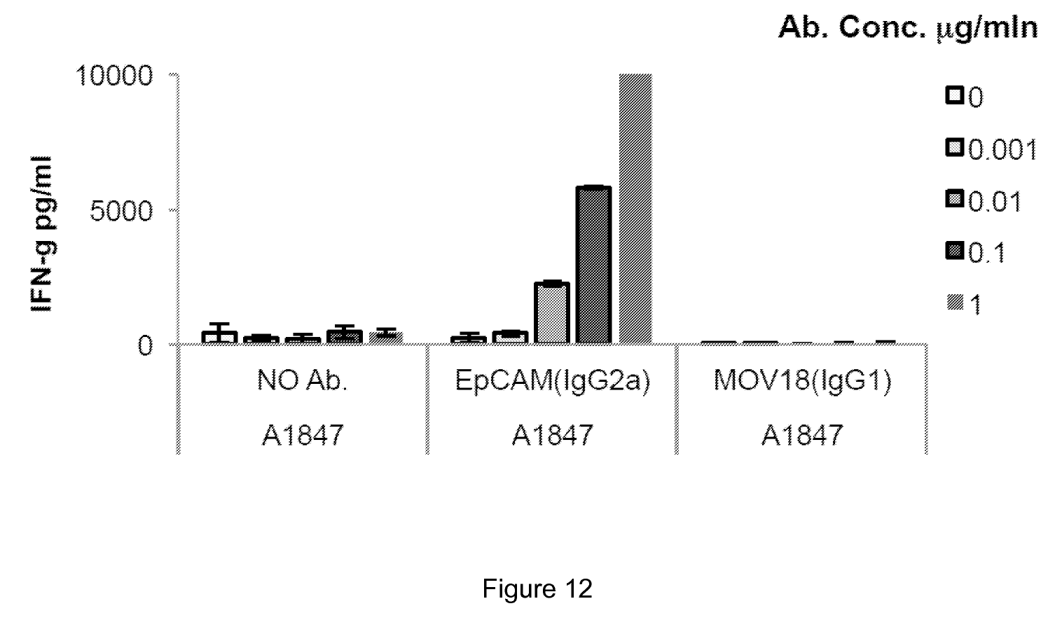

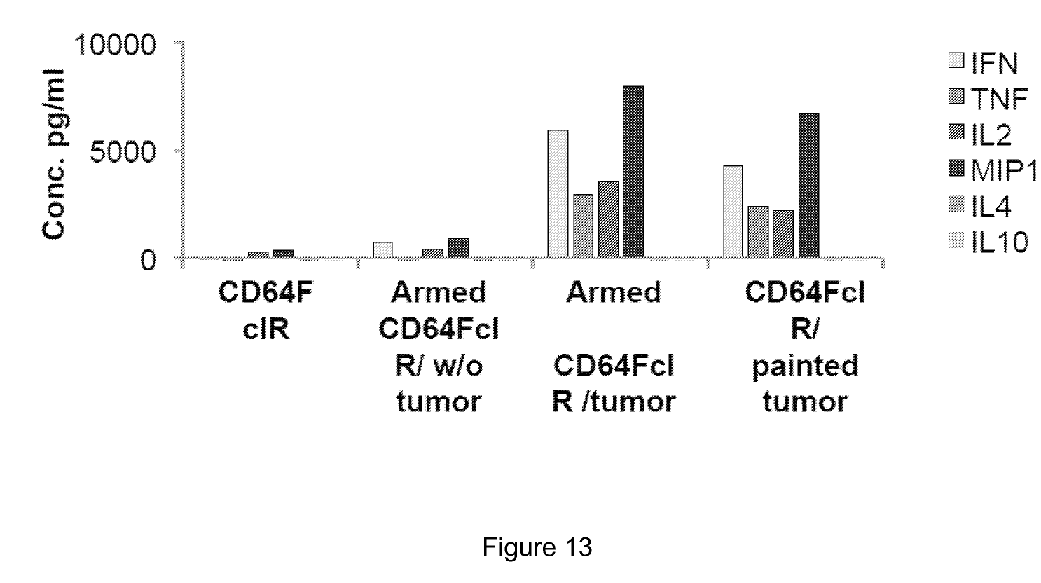

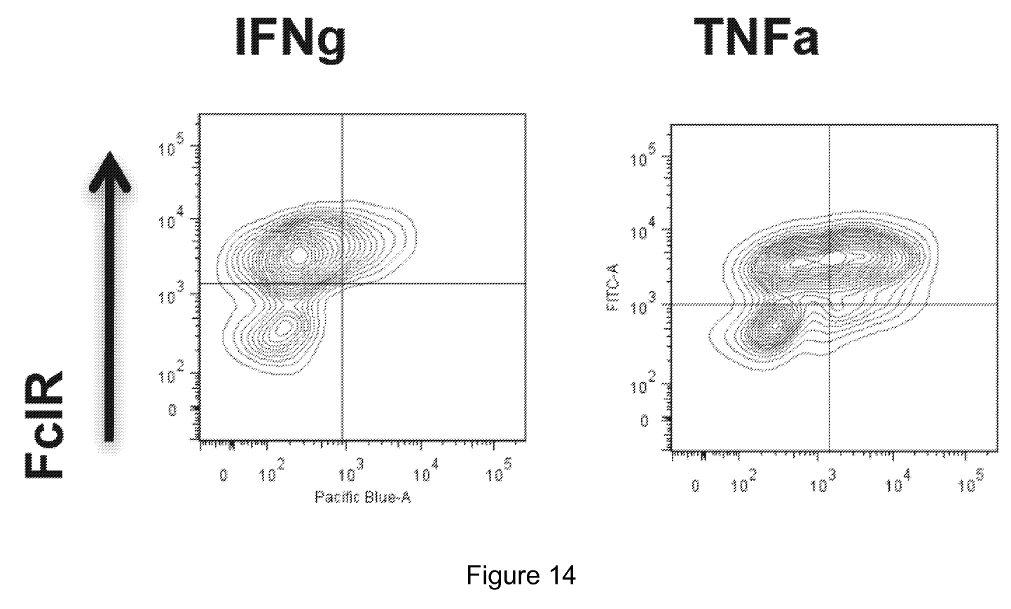

FIG. 9 shows that T cells armed with antibody Fc immune receptors specifically bound tumors displaying antigens that bind the antibodies. Likewise, T cells with Fc immune receptors specifically bound antibodies coated on the surface of target cells (FIG. 10). Upon binding and activation of the Fc immune receptor, the T cells produced IFN-γ. Both the antibody armed Fc immune receptor and Fc immune receptor binding antibodies on the surface of target cells induced the T cells to produce IFN-γ in an antibody concentration dependent manner (FIGS. 11 and 12, respectively). Also, production and secretion of various cytokines and factors (IFN-γ, TNF, IL-2, MIP1, IL-4 and IL-10) increased in T cells with activated antibody armed Fc immune receptors and Fc immune receptors bound to antibody coated target cells as compared with control cells (FIG. 13). T cells expressing both Fc immune receptors and IFN-γ (left diagram) or TNF-α are shown in FIG. 14.

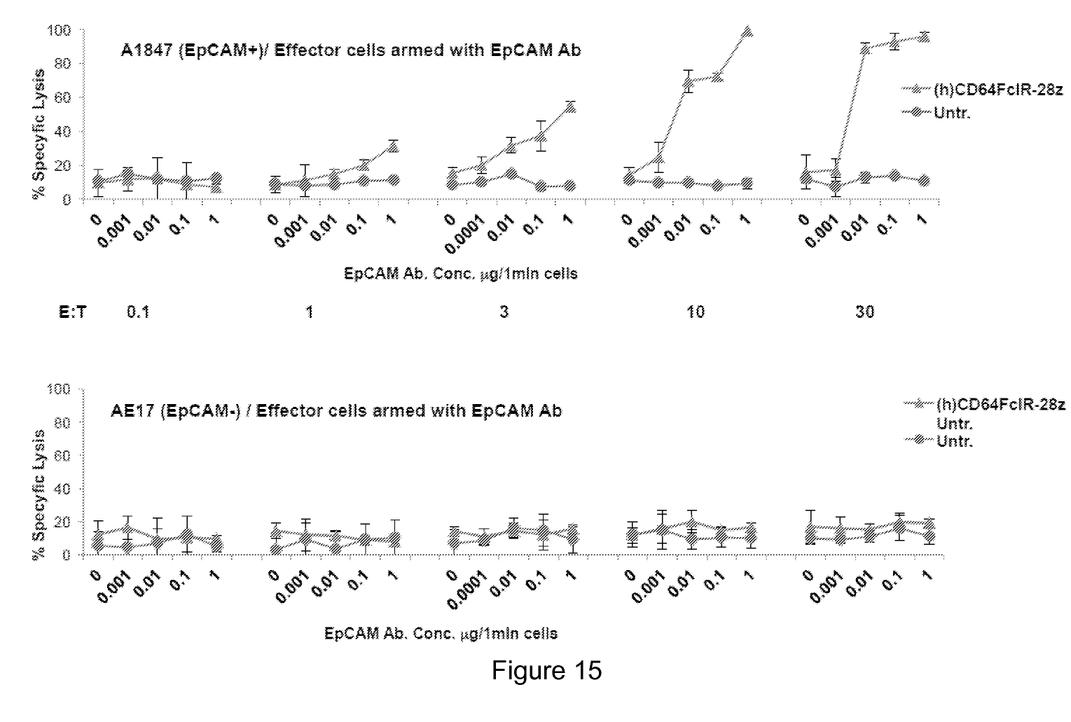

To evaluate T cell cytolytic function, CD64FcIR-28z transduced human primary T cells, or untransduced, control cells, armed with antibody at different concentrations were cultured with chromium labeled target cells, including A1847 (EpCAM+) (A) and AE17 (EpCAM−) for 4 hr (and 16 hr). Shown in FIG. 15 are percentages of lysis of target cells by chromium release at varying effector effector/target cell ratios. Anti-EpCAM antibody armed Fc immune receptor T cells had specific lytic activity against EpCAM expressing tumors (FIG. 15) through specific redirection of CD64 expressing T cells by antibody against targeted antigen (EpCAM) expressing tumors. Specific lytic activity of anti-EpCAM IgG2a armed CD64FcIR-28z T cell cytotoxicity against tumor cells was assessed by chromium release.

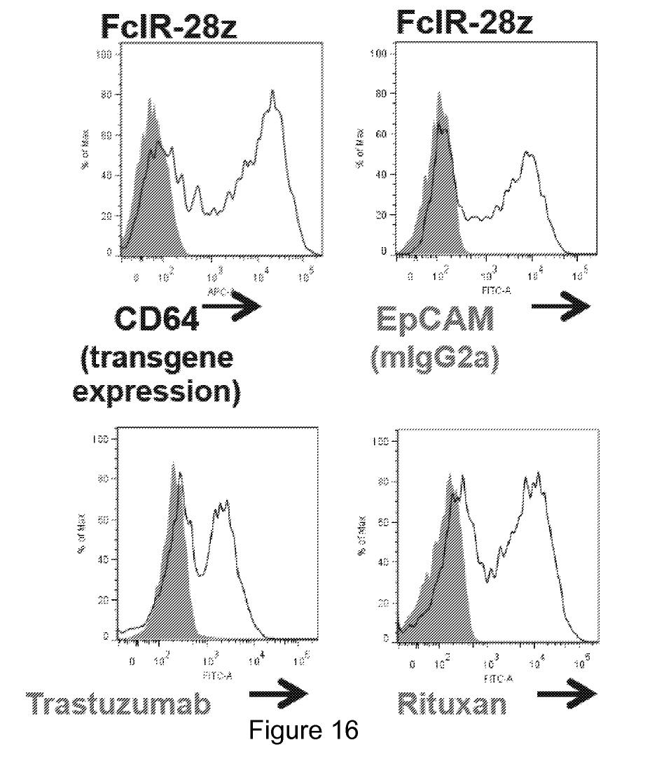

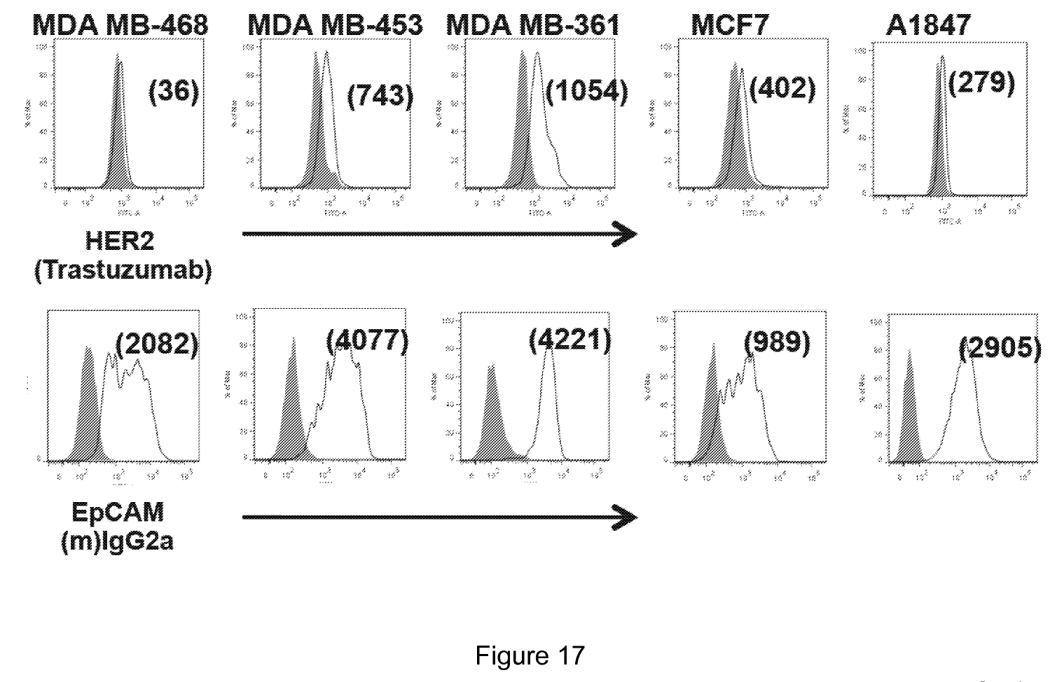

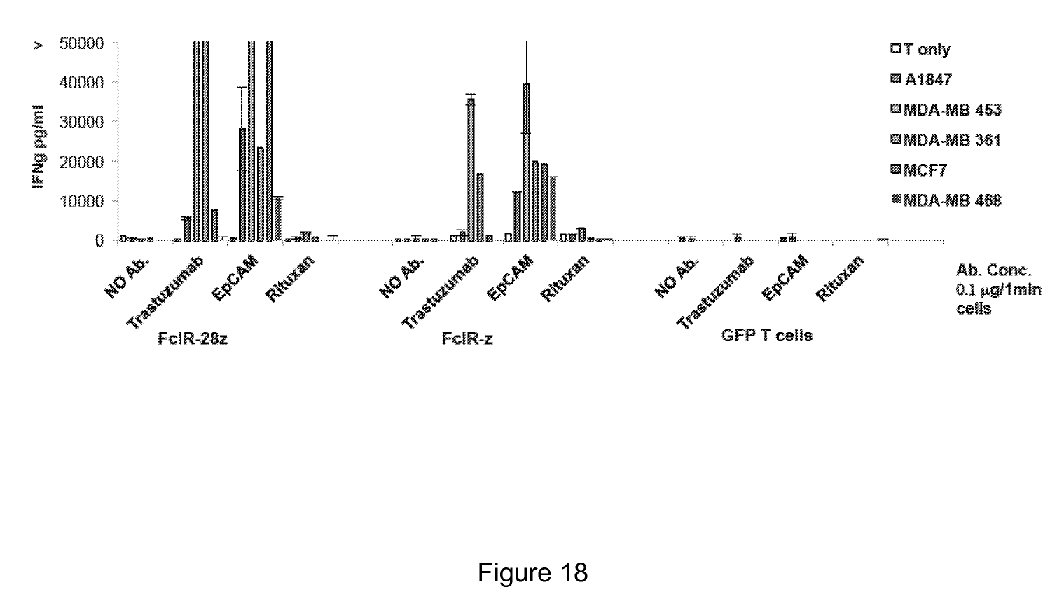

Antibody mediated immune recognition of EpCAM and Her2 expressing cancer cells by CD64 Fc immune receptors is shown in FIGS. 16 and 17. The levels of IFN-γ were increased in T cells with antibody armed Fc immune receptors for EpCAM and Her2 after immune recognition of different EpCAM and Her2 expressing cancer cells (FIG. 18).

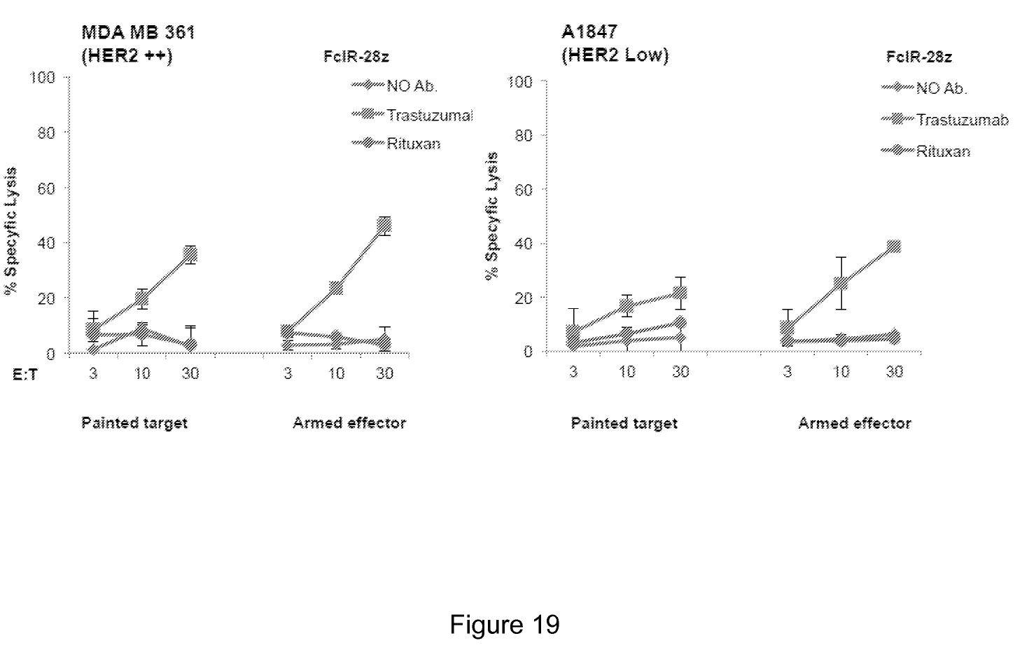

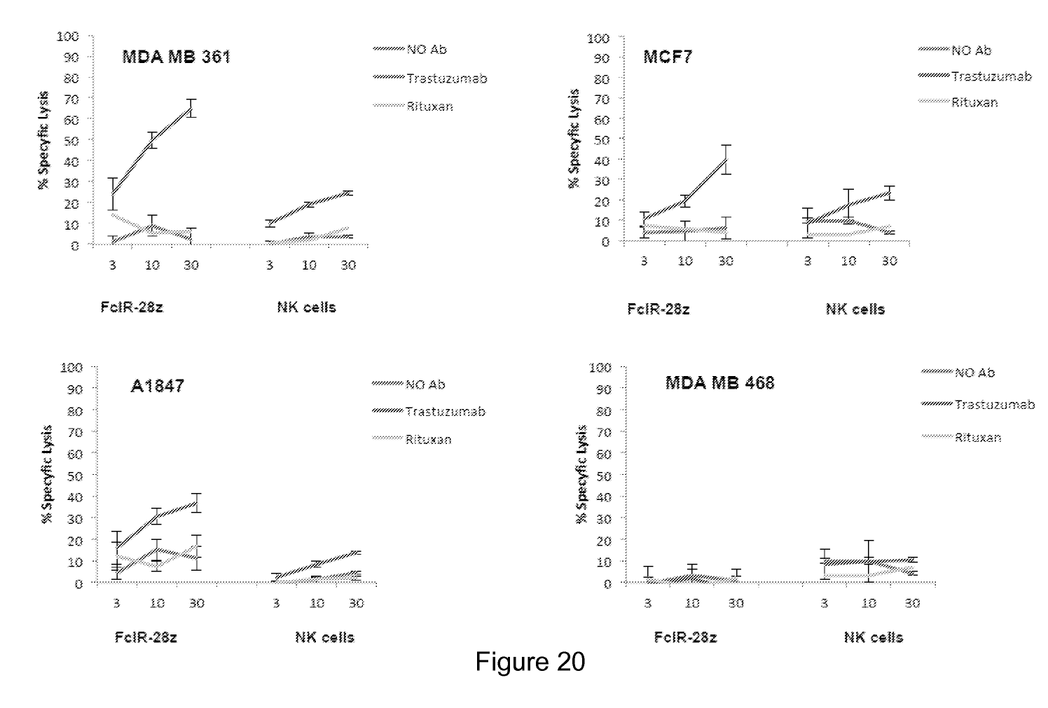

T cells expressing Fc immune receptors had similar lytic activity whether the Fc immune receptors were armed with antibody or exposed to antibody coated target cells (FIG. 19). Moreover, tumor lysis of Her2 tumor cells was significantly enhanced in the presence of Fc immune receptors (FIG. 20).

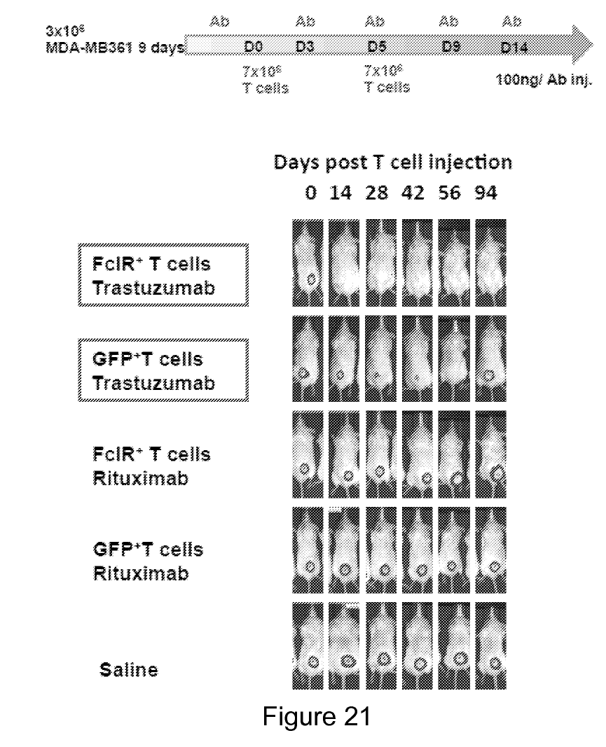

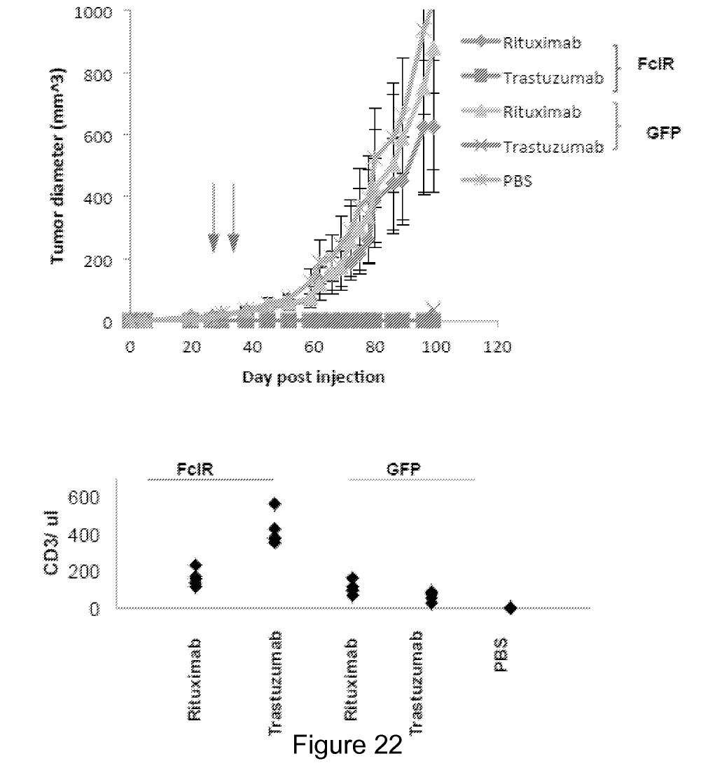

Fc immune receptor expressing T cells injected into Her2(+) tumor bearing mice had enhanced anti-tumor efficacy over Her2 antibody treatment without Fc immune receptor T cells (FIG. 21). In fact, tumor diameter was significantly decreased (upper graph of FIG. 22) with an increase in T cell persistence (lower graph of FIG. 22).

The data herein shows a proof of concept that potent T cells were designed for antibody mediated tumor therapy with a potential for no immunogenicity. The ability to use different antibodies allows for high affinity antigen specific antibodies. T cells expressing the Fc immune receptor can be tailored to the tumor or target and redirected with clinically available antibodies. Moreover, the data described herein shows improved in vitro tumor cell lysis results in comparison to conventional antibody dependent cellular cytotoxic methodologies. In summary, the Fc immune receptor shows antitumor efficacy in T cells armed with antibody or against antibody coated targets.

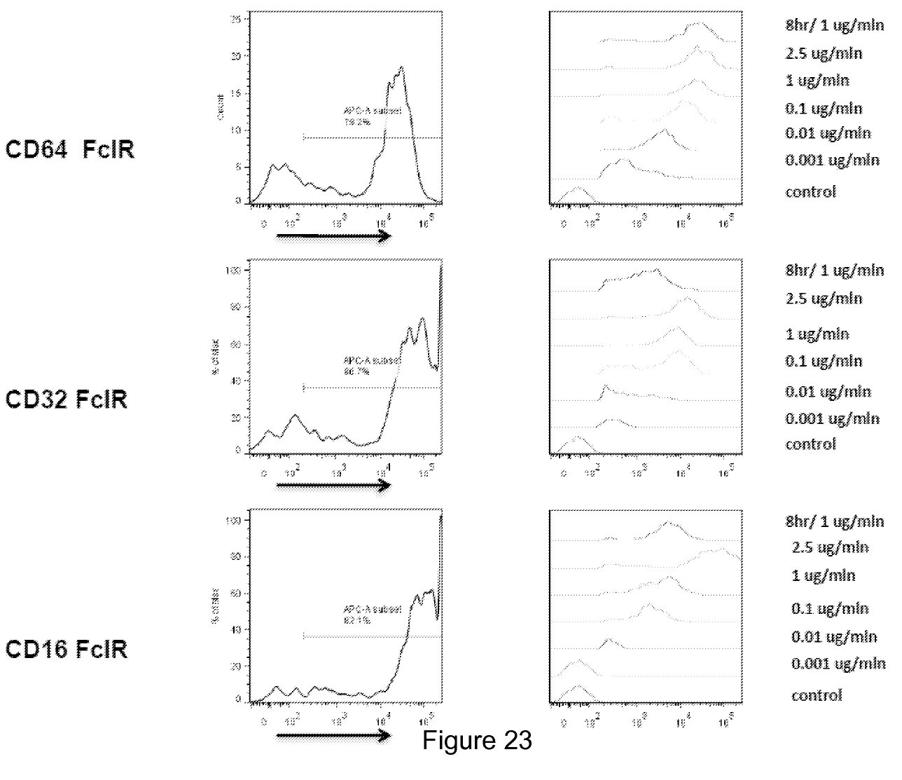

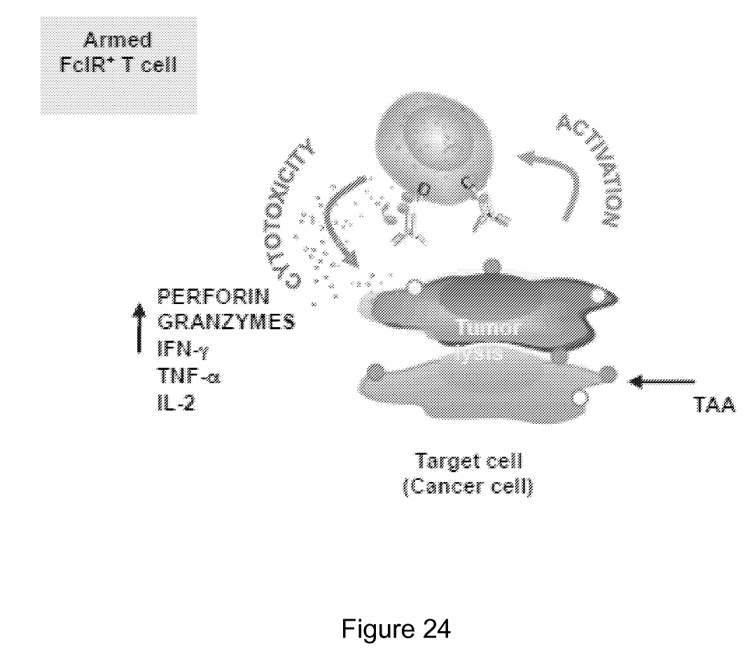

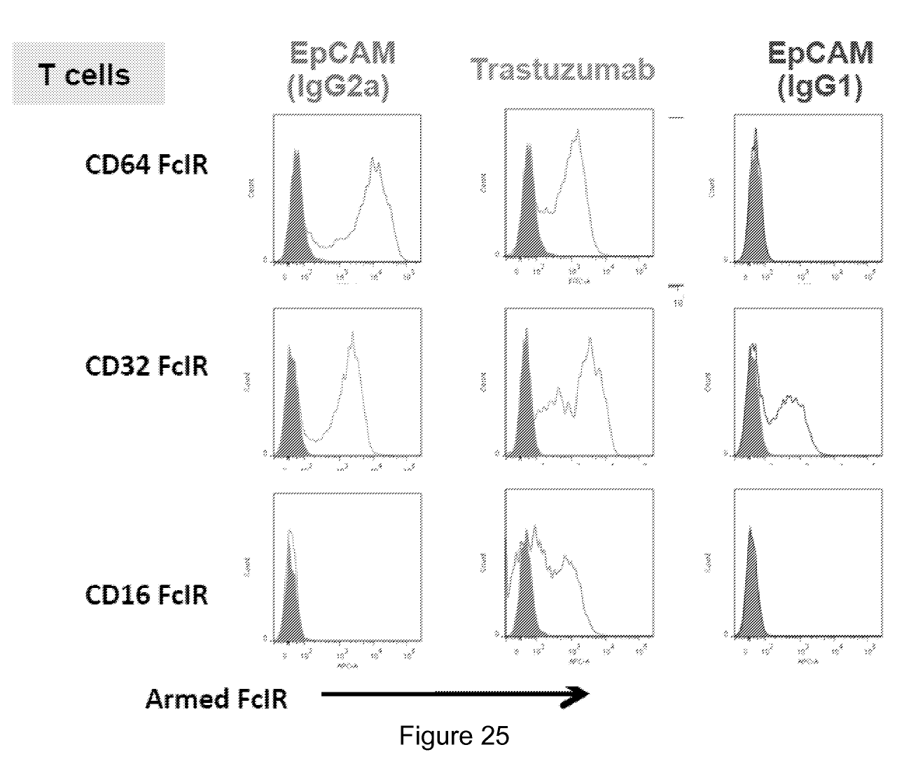

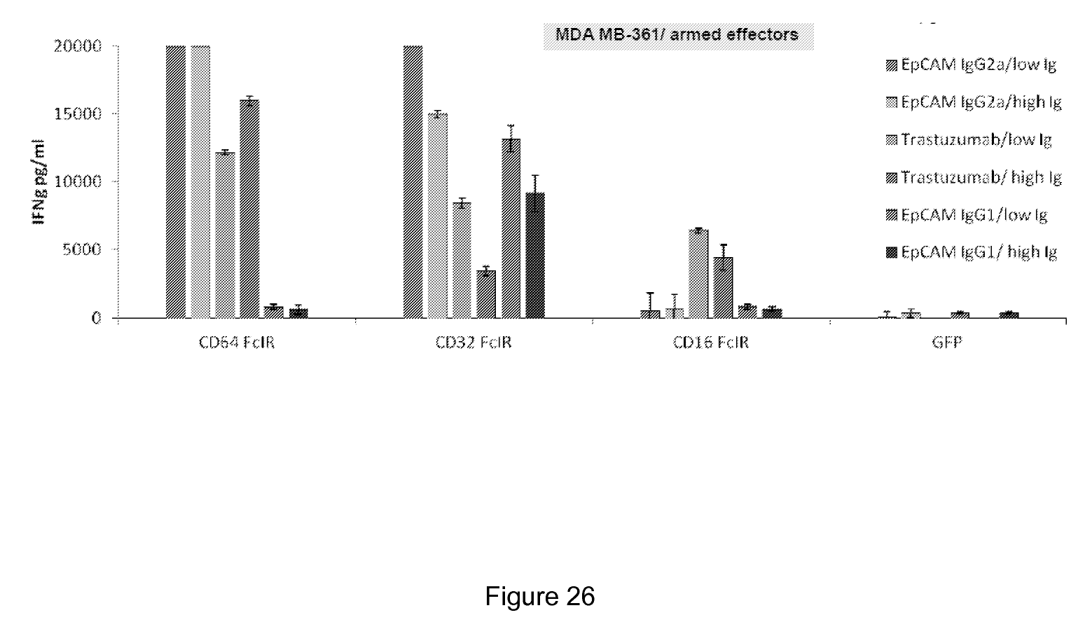

Also, Fc immune receptors with different extracellular domains (CD64, CD32 and CD16) expressed in primary T cells (FIG. 23). FIG. 24 illustrates the mechanism used to test the functional differences between Fc immune receptors with different extracellular domains. The different extracellular domains bound to EpCAM and Her2 antibodies, see FIG. 25, and T cells expressing each of the Fc immune receptors with the different extracellular domains secreted higher levels of IFN-γ levels than control T cells (FIG. 26).

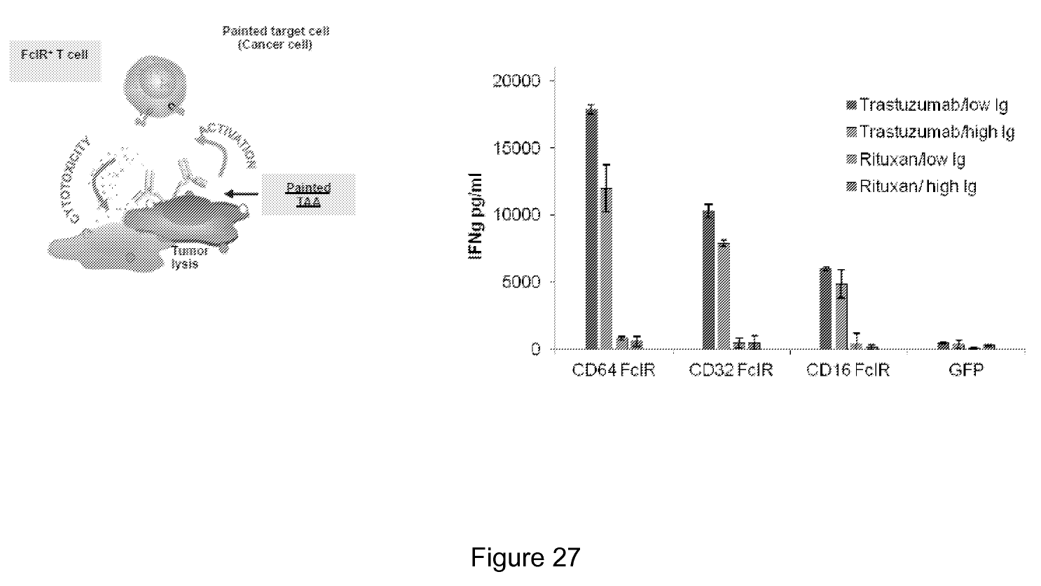

The image on the left of FIG. 27 illustrates the hypothesized mechanism to test the functional differences between Fc immune receptors with different extracellular domains that bound antibody coated target cells. The T cells expressing each of the Fc immune receptors with the different extracellular domains secreted higher levels of IFN-γ levels than control T cells (right graph in FIG. 27). Likewise, T cells expressing each of the Fc immune receptors with the different extracellular domains, either armed or contacted with antibody coated target cells, specifically bound Her2 antibodies or Her2 antibody bound Her2 expressing tumor cells.

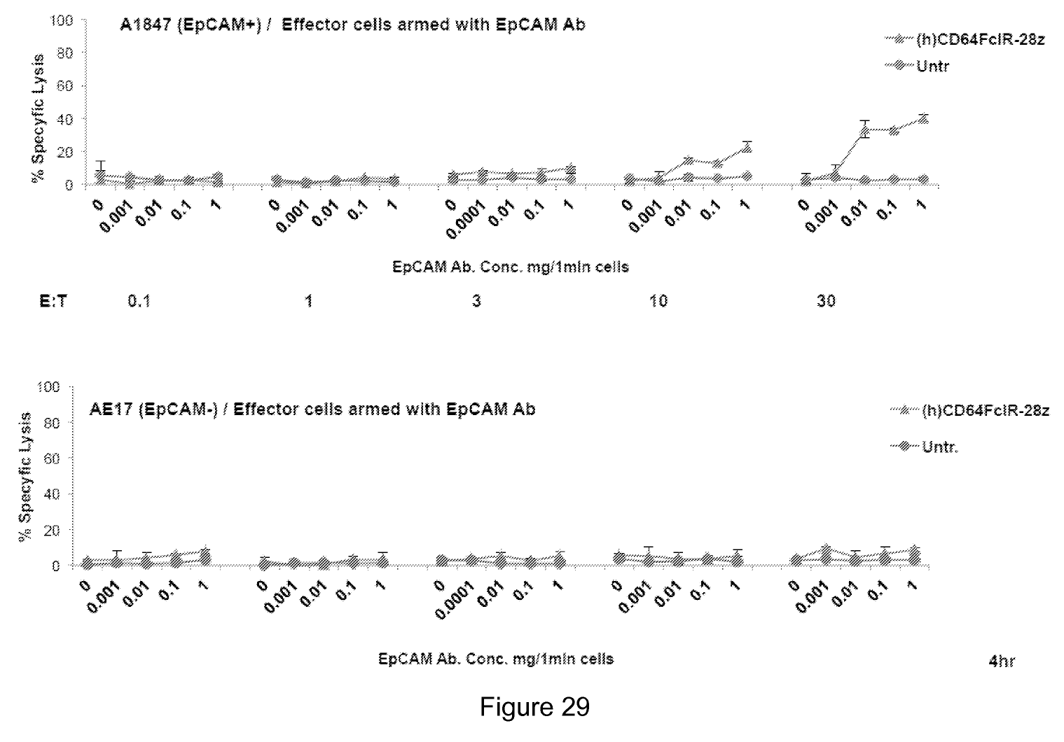

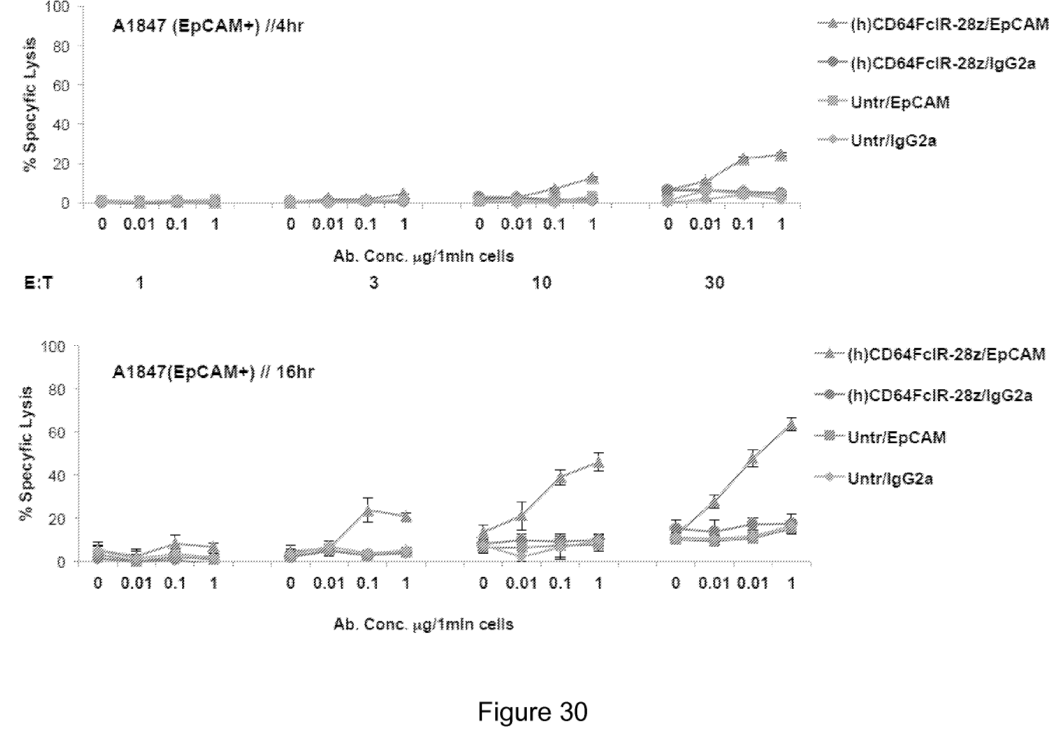

Moreover, tumor-associated antigen specific antibody armed Fc immune receptor T cells mediated tumor lysis (FIG. 29) as compared to control antibody armed Fc immune receptor T cells (FIG. 30). Specific redirection of CD64 expressing T cells with anti-EpCAM IgG2a against targeted antigen (EpCAM) expressing tumors were assayed by chromium release to determine cytotoxicity against tumor cells. CD64FcIR-28z transduced human primary T cells armed with antibody at different concentrations specifically lysed tumor cells as compared to untransduced, control cells. Lytic function of CD64Fc immune receptor expressing T cells depended on binding the armed T cells to TAA on tumor cell surfaces. Antigen specific, but not control (without specificity), antibodies redirected CD64Fc immune receptor T cells against TAA.

The immunogenicity of a cancer cell, which allows for induction of an effective immune response, depends strongly on the expression and presentation of tumor-associated antigens. However, although some tumors can be more immunogenic than others in terms of antigen expression, they may still be poorly immunogenic. One of the potential explanations can be anergy or apoptosis of tumor-specific T cells induced in the absence of costimulatory signals.

In addition, tumor cells express on their cell surface a panel of inhibitory molecules, which suppress T cell mediated immunoresponses against tumor cells (eg, CTLA4, PDL-1). This is supported by findings that after artificially induced expression of costimulatory B7 molecules, tumor cells were recognized and eliminated by activated tumor-specific T cells.

In order to expand applications for T cell-based immunotherapy and to enhance ADCC, novel effector T cells were engineered to express Fc binding immune receptors (FcIR) containing human Fc receptor of low FcγRIIIA (CD16), intermediate FcγRIIA (CD32) or high affinity FcγRI (CD64) molecules fused to intracellular TCR and co-stimulatory signaling domains in order to enable cytotoxic T cells to mediate strong mAb-directed cytotoxicity against antigen-expressing tumor cells. Following FcIR gene transduction, all forms of FcγRs efficiently expressed on the surface of primary human T cells, which allowed these cells to be armed with mAb. Trastuzumab-armed FcIR T cells specifically recognized HER2+ cancer cells, as did unarmed FcIRs but only when the cancer cells were first pre-bound with trastuzumab.

The addition of a CD28 cytoplasmic domain juxtaposed to the TCR CD3z signaling moiety increased IFN-γ secretion by FcIR-28z transduced T cells following encounter with antigen-bound mAb on the cancer cell surface. Notably, T cells expressing a high affinity FcIRI (CD64) demonstrated the greatest specific anti-tumor reactivity in comparison to cells expressing FcγRIIA (CD32) or FcγRIIIA (CD16) FcIRs. The FcIRI (CD64) T cells exhibited stronger specific lytic activity than NK cells, even at low antibody concentrations. Also, co-administration of FcIRI (CD64) FcIR-28z T cells with immunotherapeutic mAb, trastuzumab, exerted strong antitumor activity in vivo, completely eliminating HER2+ tumor.

In summary, these results show that ADCC can be enhanced by human T cells engineered to express an FcIR and that this novel approach may overcome issues of resistance to mAb-targeted therapies including those utilizing trastuzumab. Thus, enhancing the efficacy of mAbs by combination with FcIR T cell activation may have considerable therapeutic potential for a variety of malignancies, most especially for patients with impaired ADCC

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An isolated nucleic acid sequence encoding an immune receptor (IR), wherein the isolated nucleic acid sequence comprises a human nucleic acid sequence of a Fc receptor (Fc) binding domain, and a nucleic acid sequence of an intracellular domain of a costimulatory molecule.

2. The isolated nucleic acid sequence of claim 1, wherein the Fc binding domain is selected from the group consisting of CD64, CD32, CD16, a fragment thereof, and any combination thereof.

3. The isolated nucleic acid sequence of claim 1, wherein the intracellular domain comprises at least one signaling domain of the co-stimulatory molecule selected from the group consisting of CD3, CD28, a fragment thereof, and any combination thereof.

4. (canceled)

5. The isolated nucleic acid sequence of claim 1 further comprises a nucleic acid sequence of a transmembrane domain.

6. A population of cells comprising the isolated nucleic acid sequence of claim 1.

7. A vector comprising an isolated nucleic acid sequence encoding an immune receptor (IR), wherein the isolated nucleic acid sequence comprises a human nucleic acid sequence of a Fc receptor (Fc) binding domain and a nucleic acid sequence of an intracellular domain of a costimulatory molecule.

8. An isolated immune receptor (IR) comprising a Fc receptor (Fc) binding domain and an intracellular domain of a costimulatory molecule.

9. The isolated IR of claim 7, wherein the Fc binding domain is selected from the group consisting of CD64, CD32, CD16, a fragment thereof, and any combination thereof.

10. The isolated IR of claim 7, wherein the co-stimulatory molecule is selected from the group consisting of CD3, CD27, CD28, ICOS, 4-1BB, PD-1, T cell receptor (TCR), co-stimulatory molecules, any derivative or variant of these sequences, any synthetic sequence that has the same functional capability, and any combination thereof.

11. The isolated IR of claim 7 further comprising a transmembrane domain.

12. The isolated IR of claim 7, wherein the IR is capable of binding an antibody.

13. A modified T cell comprising an isolated immune receptor (IR) comprising a Fc receptor (Fc) binding domain and an intracellular domain of a costimulatory molecule.

14. The modified T cell of claim 13 further comprising an antibody bound to the Fc binding domain, wherein the antibody binds a target cell.

15. A pharmaceutical composition comprising the modified T cell of claim 13 and a pharmaceutically acceptable carrier.

16. Use of the modified T cell of claim 13 in the manufacture of a medicament for the treatment of an immune response in a subject in need thereof.

17. A method of treating a disease or condition associated with resistance to an antibody-mediated therapy in a subject comprising administering to the subject a therapeutically effective amount of a pharmaceutical composition comprising the modified T cell of claim 15.

18. A method of treating a condition in a subject, comprising administering to the subject a therapeutically effective amount of a pharmaceutical composition comprising the modified T cell of claim 15.

19. A method for stimulating a T cell-mediated immune response to a target cell or tissue in a subject comprising administering to a subject a therapeutically effective amount of a pharmaceutical composition comprising the modified T cell of claim 15.

20. A method for overcoming resistance to an antibody-mediated therapy in a subject, the method comprising administering to the subject an effective amount of a modified T cell comprising an immune receptor (IR), wherein the immune receptor comprises a Fc receptor (Fc) binding domain and an intracellular domain of a costimulatory molecule, thereby overcoming resistance to the antibody-mediated therapy in the subject.

21. A method of treating a tumor in a mammal, the method comprising administering to the subject an effective amount of a genetically modified cell comprising an immune receptor (IR), wherein the immune receptor comprises a Fc receptor (Fc) binding domain and an intracellular domain of a costimulatory molecule.

22. The method of any one of claim 20 or 21, wherein the administration comprises administering an antibody for a target cell prior to administering the effective amount of the modified T cell, and binding the modified T cell to an antibody with specificity for a target cell.

23. (canceled)
